(19) United States
(12) Patent Application Publication
(10) Pub. No.: US 2024/0168004 A1
(43) Pub. Date: May 23, 2024

(54) DESIGN AND CHARACTERIZATION OF MULTILAYERED STRUCTURES FOR SUPPORT OF LIPID BILAYERS

(71) Applicant: THE TRUSTEES OF WHEATON COLLEGE, Wheaton, IL (US)

(72) Inventors: Daniel L. Burden, Wheaton, IL (US); Lisa M. Burden, Wheaton, IL (US)

(21) Appl. No.: 17/773,485

(22) PCT Filed: Oct. 15, 2020
(86) PCT No.: PCT/US20/55763

§ 371 Date: Apr. 29, 2022

Related U.S. Application Data
(60) Provisional application No. 62/929,040, filed on Oct. 31, 2019.

(51) Int. Cl. G01N 33/487; B82Y 5/00; B82Y 15/00

(52) U.S. Cl. CPC G01N 33/48721; B82Y 5/00; B82Y 15/00; G01N 2405/00

(57) ABSTRACT

The present disclosure relates to devices having enhanced stability and durability, methods of making the same, and uses thereof. Devices of the disclosure are useful in technologies that depend on a lipid bilayer for success. In some aspects, the disclosure provides a device comprising a lipid bilayer that is linked to at least two layers of interconnected polymer filaments, wherein the lipid bilayer is attached to a substrate.

### CROSS-REFERENCE TO RELATED APPLICATIONS

This application claims the priority benefit under 35 U.S.C. § 119(e) of U.S. Provisional Patent Application No. 62/929,040, filed Oct. 31, 2019, which is incorporated herein by reference in its entirety.

### FIELD OF THE INVENTION

The present disclosure relates to devices having enhanced stability and durability, methods of making the same, and uses thereof. Devices of the disclosure are useful in technologies that depend on a lipid bilayer for success.

### BACKGROUND

Fastening a network of interconnected polymer filaments to a lipid membrane generally increases the mechanical stability of the bilayer leaflets. A number of emerging technologies critically depend on lipid bilayers and drive interest in developing arrays of highly portable and ruggedized biomembranes.¹⁻⁵ Examples include various ultrasensitive sensing applications from both the biological and solid-state nanopore literature. These include DNA sequencing,⁵⁻⁷ nucleic acid detection,⁸ digital data storage,⁹ RNA profiling,^{10, 11} protein/peptide identification,¹²⁻¹⁸ disease detection,¹⁷⁻¹⁹ synthetic polymer characterization,²⁰ small molecule detection,²¹ and ion sensing.^{22, 23} Multiple types of nanopores with various pore sizes can be utilized.^{27, 28} Additionally, research in basic scientific fields such as fundamental polymer physics,²⁰ and DNA-binding interactions,³¹ can be advanced using a rugged array of planar lipid bilayers.

### SUMMARY

The present disclosure provides devices having enhanced stability and durability, methods of making the same, and uses thereof. Accordingly, in some aspects the disclosure provides a device comprising a lipid bilayer that is linked to at least two layers of interconnected polymer filaments, wherein the lipid bilayer is attached to a substrate. In some embodiments, each of the at least two layers of interconnected polymer filaments is from about 8 to about 16 nanometers (nm) in thickness. In further embodiments, each of the at least two layers of interconnected polymer filaments bears a net positive electrostatic charge, a net negative electrostatic charge, or no electrostatic charge. In some embodiments, each of the at least two layers of interconnected polymer filaments withstands at least about 55 Pascals (Pa) of pressure without significant deformation. In further embodiments, the electrical resistivity of the device is from about 2 to about 100 gigaohms (Gohm), or from about 10 to about 100 Gohm. In some embodiments, the at least two layers of interconnected polymer filaments are chemically linked to each other. In still further embodiments, the chemical link is a covalent link, a non-covalent link, or an ionic link. In some embodiments, each of the at least two layers of interconnected polymer filaments comprises a cross-linking site. In some embodiments, from about 0.001% to about 100% of the surface of each of the at least two layers of interconnected polymer filaments comprises an anchor. In some embodiments, each of the at least two layers of interconnected polymer filaments comprises a polypeptide, an oligonucleotide, an oligosaccharide, a polymer gel, hydrogel, or a combination thereof. In further embodiments, each of the at least two layers of interconnected polymer filaments comprises a polypeptide. In some embodiments, the polypeptide is a cytoskeletal polypeptide. In further embodiments, the cytoskeletal polypeptide is a catenin, an intermediate filament protein, a microfilament protein, or a microtubule protein. In still further embodiments, the catenin is alpha catenin, beta catenin, or gamma catenin. In some embodiments, the intermediate filament protein is desmin, glial fibrillary acidic protein, keratin, nestin, or vimentin. In further embodiments, the microfilament protein is actin, actinin, filamin, gelsolin, myosin, profilin, tensin, tropomyosin, troponin, or a derivative thereof. In some embodiments, the microtubule protein is dynein, tubulin, or kinesin. In further embodiments, the at least two layers of interconnected polymer filaments are linked to each other through a cross-linking site. In some embodiments, the cross-linking site comprises (i) biotin and streptavidin; (ii) spectrin; (iii) avidin, neutravidin, or a biotin binding protein; (iv) a bridge protein from the ERM family; (v) a bridge protein from the formin family; (vi) a transmembrane glycoprotein; or (vii) digoxygenin and an antibody directed against digoxygenin. In further embodiments, the at least two layers of interconnected polymer filaments are chemically linked to the lipid bilayer. In further embodiments, the chemical link is a covalent link, a non-covalent link, or an ionic link. In some embodiments, the lipid bilayer comprises an anchor. In further embodiments, from about 0.001% to about 100% of the lipid bilayer surface comprises an anchor. In some embodiments, at least one of the at least two layers of interconnected polymer filaments are linked to the lipid bilayer through a cross-linking site. In some embodiments, the cross-linking site comprises (i) biotin and streptavidin; (ii) spectrin; (iii) avidin, neutravidin, or a biotin binding protein; (iv) a bridge protein from the ERM family; (v) a bridge protein from the formin family; (vi) a transmembrane glycoprotein; or (vii) digoxygenin and an antibody directed against digoxygenin. In some embodiments, the substrate comprises an aperture. In further embodiments, the aperture is from about 10 nanometers (nm) to about 1000 microns (μm) in diameter. In some embodiments, the substrate is a polymer resin, glass, or a semiconductor. In some embodiments, the lipid bilayer spans the aperture. In further embodiments, the aperture is about 50 microns to about 500 microns in diameter, or from about 100 nm to about 1 millimeter. In some embodiments, at least one ion channel forming at least one pore through the lipid bilayer. In further embodiments, each of the at least two layers comprises a conduit between the interconnected polymer filaments. In still further embodiments, the conduit is from about 10⁻³ to about 10⁰ microns (μm) in diameter. In some embodiments, the device comprises a plurality of apertures. In further embodiments, the device comprises one pore per aperture. In some embodiments, the device comprises about 10, or about 20, or about 30, or about 40, or about 50, or about 60, or about 70, or about 80, or about 90, or about 100, or about 200, or about 500, or about 1000, or about 2000, or about 3000, or about 5000, or about 7000, or about 10000 apertures. In some embodiments, the ion channel is a protein ion channel, Staphylococcus aureus alpha-hemolysin, Bacillus anthracis protective antigen 63, gramicidin, MspA (Mycobacterium smegmatis), OmpF porin, Kapton, OmpG, ClyA (Salmonella typhimurium), a non-naturally occurring compound, or derivatives thereof. In some embodiments, a device of the disclosure further comprises a molecular motor, wherein said motor is adjacent to the at least one pore and is capable of moving a polymer with respect to the at least one pore. In further embodiments, the molecular motor comprises a DNA polymerase, a RNA polymerase, a ribosome, an exonuclease, or a helicase and said polymer is a polynucleotide. In further embodiments, the DNA polymerase is selected from E. coli DNA polymerase I, E. coli DNA polymerase I Large Fragment (Klenow fragment), phage T7 DNA polymerase, Phi-29 DNA polymerase, Thermus aquaticus (Taq) DNA polymerase, Thermus flavus (Tfl) DNA polymerase, Thermus Thermophilus (Tth) DNA polymerase, Thermococcus litoralis (Tli) DNA polymerase, Pyrococcus furiosus (Pfu) DNA polymerase, Bacillus stearothermophilus (Bst) DNA polymerase, AMV reverse transcriptase, MMLV reverse transcriptase, and HIV-1 reverse transcriptase. In some embodiments, the RNA polymerase is selected from T7 RNA polymerase, T3 RNA polymerase, SP6 RNA polymerase, and E. coli RNA polymerase. In further embodiments, the exonuclease is selected from exonuclease Lambda, T7 Exonuclease, Exo III, RecJ1 Exonuclease, Exo I, and Exo T. In still further embodiments, the helicase is selected from E-coli bacteriophage T7 gp4 and T4 gp41 gene proteins, E. coli protein DnaB, E. coli protein RuvB, and E. coli protein rho. In some embodiments, the lipid bilayer comprises a plurality of lipid groups comprising one or more of diphytanoyl 1,2,-diacyl-sn-glycero-3-[phosphor-L-serine] (DiPHyPC), 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE). In some embodiments, each of the at least two layers of interconnected polymer filaments has a density that is from about 0.01 filaments per μm 2 to about 10,000 filaments per μm 2. In some embodiments, density of the at least two layers of interconnected polymer filaments is about the same. In some embodiments, the at least two layers of interconnected polymer filaments have different densities. In some embodiments, the device comprises three layers of interconnected polymer filaments. In further embodiments, about 2 to about 10,000 or more layers of interconnected polymer filaments. In some embodiments, the device is permeable to a molecule having a size radius of between about 50 picometers (μm) to about 500 nanometers (nm). In further embodiments, the device is permeable to a molecule having a molecular weight from about 10 to about 1,000,000 daltons. In further embodiments, the device is permeable to a molecule having a charge of from about −2×10⁶ to about +210⁶, or from about −50 to about +50.

In some aspects, the disclosure provides a method of analyzing a target polymer comprising contacting the target polymer with a device of the disclosure to allow the target polymer to move with respect to the at least one pore to produce a signal, and monitoring the signal corresponding to the movement of the target polymer with respect to the pore, thereby analyzing the target polymer. In some embodiments, the signal monitoring comprises measuring a monomer-dependent characteristic of the target polymer while the target polymer moves with respect to the pore. In further embodiments, the monomer dependent property is the identity of a monomer or the number of monomers in the polymer. In some embodiments, the method further comprises altering the rate of movement of the polymer before, during, or after the signal monitoring. In some embodiments, the target polymer is an oligonucleotide, a polypeptide, or an oligosaccharide. In further embodiments, oligonucleotide is DNA. In some embodiments, the analyzing comprises a chemical characterization. In some embodiments, the chemical characterization is a characterization of DNA, a synthetic polymer, a small molecule, or an ion. In some embodiments, the characterization of DNA comprises nucleotide sequencing or genotyping.

In some aspects, the disclosure provides a method of forming a device comprising: (a) providing a lipid bilayer, the lipid bilayer comprising a first anchor, wherein the lipid bilayer is associated with a substrate, the substrate comprising an aperture and an electrode; (b) applying a linker molecule; (c) providing a first layer of polymer filaments comprising a second anchor, thereby creating a cross-linking site between the first anchor, the linker molecule, and the second anchor, thereby linking the lipid bilayer to the first layer of polymer filaments; (d) applying the linker molecule; (e) providing a second layer of polymer filaments comprising a third anchor, thereby creating a cross-linking site between the second anchor, the linker molecule, and the third anchor, thereby linking the first layer of polymer filaments to the second layer of polymer filaments; and (f) inserting a pore into the lipid bilayer, thereby forming the device. In some embodiments, the method further comprises applying the linker molecule between steps (e) and (f); and providing a third layer of polymer filaments comprising a fourth anchor, thereby creating a cross-linking site between the third anchor, the linker molecule, and the fourth anchor, thereby linking the second layer of polymer filaments to the third layer of polymer filaments. In some embodiments, each of the first layer of polymer filaments and the second layer of polymer filaments is from about 8 to about 16 nanometers (nm) in thickness. In further embodiments, the third layer of polymer filaments is from about 8 to about 16 nanometers (nm) in thickness. In some embodiments, each of the first layer of polymer filaments and the second layer of polymer filaments withstands at least about 55 Pascals (Pa) of pressure without significant deformation. In some embodiments, the third layer of polymer filaments withstands at least about 55 Pascals (Pa) of pressure without significant deformation. In some embodiments, the electrical resistivity of the device is from about 10 to about 100 gigaohms (Gohm). In further embodiments, from about 0.001% to about 100% of the surface of each of the first layer of polymer filaments, the second layer of polymer filaments, and the third layer of polymer filaments each comprises an anchor. In some embodiments, the first layer of polymer filaments, the second layer of polymer filaments, and the third layer of polymer filaments each comprises a polypeptide, an oligonucleotide, an oligosaccharide, a polymer gel, hydrogel, or a combination thereof. In some embodiments, the first layer of polymer filaments, the second layer of polymer filaments, and/or the third layer of polymer filaments each comprises a polypeptide. In further embodiments, the polypeptide is a cytoskeletal polypeptide. In still further embodiments, the cytoskeletal polypeptide is a catenin, an intermediate filament protein, a microfilament protein, or a microtubule protein. In still further embodiments, the catenin is alpha catenin, beta catenin, or gamma catenin. In yet additional embodiments, the intermediate filament protein is desmin, glial fibrillary acidic protein, keratin, nestin, or vimentin. In some embodiments, the microfilament protein is actin, actinin, filamin, gelsolin, myosin, profilin, tensin, tropomyosin, troponin, or a derivative thereof. In some embodiments, the microtubule protein is dynein, tubulin, or kinesin. In some embodiments, the first, second, third, and fourth anchors are the same. In some embodiments, the first, second, third, and fourth anchors comprise biotin, spectrin, a bridge protein from the ERM family, a bridge protein from the formin family, a transmembrane glycoprotein, digoxygenin, or a combination thereof. In some embodiments, the linker molecule is streptavidin, avidin, neutravidin, a biotin binding protein, an antibody directed against digoxygenin, or a combination thereof. In some embodiments, the aperture is from about 100 nanometers (nm) to about 1000 microns (μm) in diameter. In some embodiments, the substrate is a polymer resin, glass, or a semiconductor. In some embodiments, the lipid bilayer spans the aperture. In further embodiments, the polymer filaments of each of the first layer and the second layer are separated by a conduit. In some embodiments, the polymer filaments of the third layer are separated by a conduit. In some embodiments, the conduit is from about 10⁻³ to about 10⁰ microns (μm) in diameter. In some embodiments, the lipid bilayer comprises a plurality of lipid groups comprising one or more of diphytanoyl 1,2,-diacyl-sn-glycero-3-[phosphor-L-serine] (DiPHyPC), 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE). In some embodiments, the first layer of polymer filaments, the second layer of polymer filaments, and the third layer of polymer filaments each has a density that is from about 0.01 filaments per lie to about 10,000 filaments per lie. In some embodiments, density of each of the first layer of polymer filaments, the second layer of polymer filaments, and the third layer of polymer filaments is about the same. In some embodiments, one or more of the first layer of polymer filaments, the second layer of polymer filaments, and the third layer of polymer filaments have different densities. In some embodiments, the surface charge density of the first layer of polymer filaments, the second layer of polymer filaments, and the third layer of polymer filaments is controlled by adjusting the pH of the buffer. In some embodiments, the surface charge density of the first layer of polymer filaments, the second layer of polymer filaments, and the third layer of polymer filaments is controlled by adjusting the ionic strength of the buffer. In some embodiments, the formation success frequency is from about 70% to about 90% or more.

In some aspects, the disclosure provides a method of forming a device comprising: (a) providing a lipid bilayer comprising biotin, wherein the lipid bilayer is associated with a substrate, the substrate comprising an aperture and an electrode; (b) applying avidin; (c) providing a first layer of polymer filaments comprising biotin, thereby creating a cross-linking site between the biotin on the lipid bilayer, the avidin, and the biotin on the first layer of polymer filaments, thereby linking the lipid bilayer to the first layer of polymer filaments; (d) applying avidin; (e) providing a second layer of polymer filaments comprising biotin, thereby creating a cross-linking site between the biotin on the first layer of polymer filaments, the avidin, and the biotin on the second layer of polymer filaments, thereby linking the first layer of polymer filaments to the second layer of polymer filaments; and (f) inserting a pore into the lipid bilayer, thereby forming the device. In some embodiments, the method further comprises applying avidin between steps (e) and (f), and providing a third layer of polymer filaments comprising biotin, thereby creating a cross-linking site between the biotin on the second layer of polymer filaments, the avidin, and the biotin on the third layer of polymer filaments, thereby linking the second layer of polymer filaments to the third layer of polymer filaments.

### BRIEF DESCRIPTION OF THE FIGURES

FIG. 1 shows that Microelectrode Cavity Array (MECA4-Fluo) chips allow four independent lipid bilayers to be formed simultaneously. (A) The liquid filled chamber, electrodes, and electrical contacts are packaged in a compact chip format with five exposed electrical contacts located at the top of the chip. (B) Magnified view of the four microcavities and associated printed circuit elements. (C) Cross-section illustration of a single microcavity with a suspended lipid bilayer and an inserted nanopore. Aqueous solution fills the microcavity and resides on both sides of the bilayer.

FIG. 2 shows (A) The Orbit Mini contains all electronics necessary for probing bilayers and nanopores formed on a MECA4-Fluo chip. The unit connects to a laptop computer, where voltage and current data are stored and processed, through a USB cable. (B) A fluorescence microscope enables simultaneous optical and electrical measurements in conjunction with the Orbit Mini.

FIG. 3 shows the perfusion system mounted on 3D micropositioners that allowed for rapid solution exchange of array chip.

FIG. 4 shows that creating actin filament layers involves an iterative procedure (A-wash-Bₙ-wash)_{N}, where A is a linker, B is F-actin, wash indicates a rinse of the chamber, n designates a sub-saturation injection of F-actin, and N is the total number of linked layers formed in the multilayer structure.

FIG. 5 depicts that TIRF microscopy enables imaging near the bilayer surface with high contrast because the evanescent wave only penetrates approximately 500 nm beyond the glass boundary.

FIG. 6 shows TIRF images from glass supported bilayers: (A) Single-layered structure (N=1) linked using avidin on a bilayer containing 0.1% biotinylated lipids. (B) Two-layered (N=2) structure using avidin to cross-link filament layers. (C) Three-layered (N=3) structure with avidin cross-linking. The spatially averaged fluorescence intensity for the images grew monotonically with the number of added cross-linked layers (N).

FIG. 7 shows that long-range electrostatic forces dictate the extent of filament deposition and help bind layers together: (A) F-actin binds to avidin-coated bilayers with high affinity under low salt (50 mM); (B) Charge screening under high salt conditions (500 mM) prohibited surface binding; (C) A return to low salt concentration restored filament deposition.

FIG. 8 shows (A) A single MECA4-Fluo microcavity with singled-layered structure attached to the bilayer (N=1) suspended over the aperture. Outline of the 150 μm cavity rim is highlighted; (B) A single MECA4-Fluo microcavity with a multilayer structure attached to the bilayer (N=3) suspended over the microcavity aperture. Filament density is notably increased from N=1 image.

FIG. 9 shows the convex curvature observed in some multilayered structures formed on a MECA4-Fluo microcavity. Intertwined filaments of the multilayered structure are visible along the portion of the contour in the object focal plane.

FIG. 10 shows an axial scan of MECA4-Fluo cavity with an N=3 multilayer structure. Images from various depths within the cavity are shown with intensity levels autoscaled within each image: (A) cavity bottom, 0.0 μm, Ag/AgCl electrode surface; (B)+7 μm, center of cavity. No distinct filaments are visible; (C)+13 μm, multilayered structure spanning the microcavity aperture evidenced by filamentous texture and partially photobleached region; (D)+19 μm, out of focus multilayered structure. Image sequence demonstrates the presence of a thin suspended structure with aqueous solution residing on either side of the bilayer.

FIG. 11 shows typical WT αHL channel insertions with an uncoated lipid bilayer (A) and a bilayer coated with a multilayered structure (N=3) (B). Insertions demonstrate that the multilayer structure does not prohibit protein migration to the lipid surface and channel assembly.

FIG. 12 shows the number of single-molecule diffusion trajectories before and after BSA addition. BSA is added to a multilayer structure (N=3) at approximately 0.5 s (+BSA) and is followed by an immediate increase in surface-bound molecules that diffuse laterally. The background number of trajectories after photobleaching, but prior to BSA addition, is overlaid for reference (dots).

### DETAILED DESCRIPTION

Planar lipid bilayers play a central role in nanopore sensing and ion channel electrophysiology. However, bilayer fragility often limits applications. The present disclosure provides multilayered devices that can be cross-linked to arbitrary thicknesses while retaining an open network assembly that enables rapid diffusive permeability.

The term “membrane” as used herein refers to a composite structure comprising a lipid bilayer and a substrate. Therefore, as used herein, a “membrane” comprises a lipid bilayer and a substrate.

As used herein, the term “cavity” or “microcavity” refers to a depression in the substrate. The term “aperture” as used herein refers to the perimeter, or rim, located at the top of the cavity/microcavity. In this regard, an aperture is a feature possessed by all cavities/microcavities.

As used herein, the term “pore” is used interchangeably with the term “nanopore” and refers to an ion-channel molecule.

As used herein the term “conduit(s)” refers to a system of three-dimensional, highly branched, and interconnected tunnels, or passageways, that connect bulk solution to the lipid bilayer surface. The walls of the “conduits” are defined by (or formed by) interconnected polymer filaments and linker molecules. A “conduit” is generally filled with movable water and solute molecules that connect bulk solution located beyond the multilayer structure, to a small portion of the lipid bilayer surface. Thus, “conduits” pass through multiple layers of support structure, including a linker molecule layer (type A), a filament layer (type B), and multiple cross-linked composite structures (ABₙ)_{N}.

The terms “cross-linking site,” “linker molecule,” and “anchor” are used throughout the disclosure. A “cross-linking site” comprises two anchors originating from two adjacent layers that are bridged by a linker molecule. Thus, a cross-linking site is constructed from an anchor-linker molecule-anchor connection.

An “anchor” as used herein refers to a covalently-joined chain of atoms that is terminated by a “key” moiety (e.g., biotin). A “linker molecule” is a molecule that possesses one or more “lock” sites that can bind to the key moiety on an anchor. Inserting a key moiety into a lock site enables a linker molecule to act as a bridge between layers. In any of the aspects or embodiments of the disclosure, linker molecules are joined to key moieties via non-covalent bonds, whereas an anchor is joined to a lipid bilayer or to a polymer filament layer via a covalent bond. Thus, a device of the disclosure employs a combination of both covalent bonds and non-covalent bonds. It is contemplated that any type of bond may be used to join a linker molecule to an anchor. In some aspects, linker molecules join layers together through two or more anchors, wherein each anchor originates from a different layer. In some embodiments, interlayer connections are generated via electrostatic forces, ionic bonds, and hydrogen bonds in addition to the interlayer connection generated via one or more cross-linking sites. Examples of linker molecules include, but are not limited to, streptavidin, avidin, neutravidin, a biotin binding protein, an antibody directed against digoxygenin, or a combination thereof. Examples of key moieties include, but are not limited to, biotin and digoxygenin.

Unless otherwise defined herein, scientific and technical terms employed in the disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise.

### Devices

In any aspects or embodiments of the disclosure, a device is provided wherein the device comprises a lipid bilayer that is linked to at least two layers of interconnected polymer filaments, wherein the lipid bilayer is further attached to a substrate.

Polymer filaments. According to the disclosure, and in various embodiments, each of the at least two layers of interconnected polymer filaments comprises a polypeptide, an oligonucleotide, an oligosaccharide, a polymer gel, hydrogel, or a combination thereof. In various embodiments, at least one of the at least two layers of interconnected polymer filaments is chemically linked to the bilayer (e.g., lipid bilayer) of the device. In further embodiments, the chemical link is a covalent link. In some embodiments, the link is a non-covalent link.

In some embodiments, each of the at least two layers of interconnected polymer filaments is from about 5 to about 20 nanometers (nm) in thickness. In further embodiments, each of the at least two layers of interconnected polymer filaments is from about 5 to about 10, or from about 5 to about 7, or from about 8 to about 20, or from about 8 to about 16, or from about 8 to about 10 nanometers (nm) in thickness. In further embodiments, each of the at least two layers of interconnected polymer filaments is, is about, or is at least about 5, 6, 7, 8, 9, 10, 11, 12, 13, 14, 15, 16, 17, 18, 19, or 20 nanometers (nm) in thickness.

The disclosure also contemplates that each of the at least two layers of interconnected polymer filaments bears a positive electrostatic charge, a negative electrostatic charge, or no electrostatic charge. These aspects of creating a device in which each layer bears excess charge or is charge neutral, and combining those layers in various ways, gives rise to the unique observation of ion-selective permeability that is further discussed herein below.

By virtue of the design of the devices of the disclosure, each layer of polymer filaments is also contemplated to provide exceptional support to the device. Thus, in various embodiments, each of the at least two layers of interconnected polymer filaments withstands about or at least about 55 Pascals (Pa) of pressure without significant deformation. In further embodiments, each of the at least two layers of interconnected polymer filaments withstands about or at least about 60, 65, 70, 75, 80, 85, 90, 95, 100, 105, 110, 115, 120, 125, 130, 135, 140, 145, 150, 155, 160, 165, 170, 175, 180, 185, 190, 195, or 200 Pascals (Pa) of pressure without significant deformation. “Significant deformation” can be quantified in terms of percentage change in capacitance, which is directly proportional to a change in bilayer area. According to the disclosure, a 5% change or more in capacitance or area at 100 Pa of applied pressure is considered a significant deformation. Strictly by way of example, at 100 Pa of applied pressure, devices of the disclosure generally demonstrate less than a 5% change in capacitance or area. This is better performance than demonstrated by a slab hydrogel approach (Malmstadt et al., Adv. Mater. 2008, 20, 84-89) which is a non-layered, thick structure that breaks at approximately 55 Pa.

The disclosure also contemplates that the electrical resistivity of the device is from about 2 to about 100 gigaohms (Gohm), or from about 10 to about 100 Gohm. In further embodiments, the electrical resistivity of a device of the disclosure is from about 5 to about 100, or from about 5 to about 90, or from about 5 to about 80, or from about 5 to about 70, or from about 5 to about 60, or from about 5 to about 50, or from about 5 to about 40, or from about 50 to about 30, or from about 5 to about 20, or from about 5 to about 10, or from about 10 to about 100, or from about 10 to about 90, or from about 10 to about 80, or from about 10 to about 70, or from about 10 to about 60, or from about 10 to about 50, or from about 10 to about 40, or from about 10 to about 30, or from about 10 to about 20 Gohm. In still further embodiments, the electrical resistivity of a device of the disclosure is, is about, or is at least about 2, 3, 4, 5, 10, 20, 30, 40, 50, 60, 70, 80, 90, or 100 Gohm.

The density of each of the at least two layers of interconnected polymer filaments may be varied. Adjusting the density of a layer of polymer filaments is advantageous, for example, to create a device that is permeable to a molecule of a particular size. Aspects of tuning a device of the disclosure are further described herein below. In various embodiments, each of the at least two layers of interconnected polymer filaments has a density that is from about 0.01 filaments per um² to about 10,000 filaments per um². In further embodiments, each of the at least two layers of interconnected polymer filaments has a density that is from about 0.01 filaments per um² to about 9,000 filaments per um², or from about 0.01 filaments per um² to about 8,000 filaments per um², or from about 0.01 filaments per um² to about 7,000 filaments per um², or from about 0.01 filaments per um² to about 6,000 filaments per um², or from about 0.01 filaments per um² to about 5,000 filaments per um², or from about 0.01 filaments per um² to about 4,000 filaments per um², or from about 0.01 filaments per um² to about 3,000 filaments per um², or from about 0.01 filaments per um² to about 2,000 filaments per um², or from about 0.01 filaments per um² to about 1,000 filaments per um², or from about 10 filaments per um² to about 10,000 filaments per um², or from about 10 filaments per um² to about 9,000 filaments per um², or from about 10 filaments per um² to about 8,000 filaments per um², or from about 10 filaments per um² to about 7,000 filaments per um², or from about 10 filaments per um² to about 6,000 filaments per um², or from about 10 filaments per um² to about 5,000 filaments per um², or from about 10 filaments per um² to about 4,000 filaments per um², or from about 10 filaments per um² to about 3,000 filaments per um², or from about 10 filaments per um² to about 2,000 filaments per um², or from about 10 filaments per um² to about 1,000 filaments per um². In further embodiments, each of the at least two layers of interconnected polymer filaments has a density that is, is about, or is at least about 0.01, 0.05, 1, 5, 10, 20, 30, 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, or 10000 filaments per um². In further embodiments, each of the at least two layers of interconnected polymer filaments has a density that is less than or is less than about 1, 5, 10, 20, 30, 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, or 10000 filaments per um². In some embodiments, density of the at least two layers of interconnected polymer filaments is about the same. In further embodiments, the at least two layers of interconnected polymer filaments have different densities.

Regarding permeability of the devices of the disclosure, it is contemplated in various embodiments that a device as disclosed herein can be generated that is permeable to molecules of various size radii, molecules having various molecule weights, and molecules having various charges. Accordingly, in some embodiments, a device of the disclosure is permeable to a molecule having a size radius of between about 50 picometers (μm) to about 500 nanometers (nm). In further embodiments, a device of the disclosure is permeable to a molecule having a size radius of between about 50 μm to about 400 nm, or between about 50 μm to about 300 nm, between about 50 μm to about 200 nm, between about 50 μm to about 100 nm, between about 50 μm to about 50 nm, between about 50 μm to about 900 μm, between about 50 μm to about 800 μm, between about 50 μm to about 700 μm, between about 50 μm to about 600 μm, between about 50 μm to about 500 μm, between about 50 μm to about 400 μm, between about 50 μm to about 300 μm, between about 50 μm to about 200 μm, or between about 50 μm to about 100 μm. In various embodiments, a device of the disclosure is permeable to a molecule having a size radius that is, is about, or is at least about 50 μm, 100 μm, 200 μm, 500 μm, 700 μm, 1 nm, 5 nm, 10 nm, 50 nm, 70 nm, 100 nm, 200 nm, or 500 nm.

In further embodiments, a device of the disclosure is permeable to a molecule having a molecular weight from about 10 to about 1,000,000 daltons, or from about 10 to about 900,000 daltons, from about 10 to about 800,000 daltons, from about 10 to about 700,000 daltons, from about 10 to about 600,000 daltons, from about 10 to about 500,000 daltons, from about 10 to about 400,000 daltons, from about 10 to about 300,000 daltons, from about 10 to about 300,000 daltons, from about 10 to about 100,000 daltons, from about 10 to about 90,000 daltons, from about 10 to about 80,000 daltons, from about 10 to about 70,000 daltons, from about 10 to about 60,000 daltons, from about 10 to about 50,000 daltons, from about 10 to about 40,000 daltons, from about 10 to about 30,000 daltons, from about 10 to about 20,000 daltons, from about 10 to about 10,000 daltons, from about 10 to about 9,000 daltons, from about 10 to about 8,000 daltons, from about 10 to about 7,000 daltons, from about 10 to about 6,000 daltons, from about 10 to about 5,000 daltons, from about 10 to about 4,000 daltons, from about 10 to about 3,000 daltons, from about 10 to about 2,000 daltons, from about 10 to about 1,000 daltons. In further embodiments, a device of the disclosure is permeable to a molecule having a molecular weight that is, is about, or is at least about 10, 50, 100, 200, 500, 700, 1000, 2000, 5000, 10000, 20000, 50000, 70000, 100000, 200000, 500000, 700000, or 1000000 daltons. In some embodiments, a device of the disclosure is permeable to a molecule (e.g., a nucleic acid) having a charge of from about −2×10⁶ to about +2×10⁶ or more. In further embodiments, a device of the disclosure is permeable to a molecule having a charge of about −50 to about +50. In still further embodiments, a device of the disclosure is permeable to a molecule having a charge of about −50, −40, −30, −20, −10, 0, +10, +20, +30, +40, or +50.

In any of the aspects or embodiments of the disclosure, the at least two layers of interconnected polymer filaments are chemically linked to each other. In any of the aspects or embodiments of the disclosure, at least one of the at least two layers of interconnected polymer filaments is chemically linked to the lipid bilayer. In various embodiments, the chemical link to the polymer filament and/or to the lipid bilayer is a covalent bond, a non-covalent interaction (e.g., van der Waals interactions, steric interactions, pi-pi stacking, electrostatic attractions, hydrogen bonding), or an ionic bond. In some embodiments, the device comprises three layers of interconnected polymer filaments. In further embodiments, the device comprises from about 2 to about 10,000 or more layers of interconnected polymer filaments. In further embodiments, the device comprises from about 2 to about 9,000, or from about 2 to about 8,000, or from about 2 to about 7,000, or from about 2 to about 6,000, or from about 2 to about 5,000, or from about 2 to about 4,000, or from about 2 to about 3,000, or from about 2 to about 2,000, or from about 2 to about 1,000, or from about 2 to about 900, or from about 2 to about 800, or from about 2 to about 700, or from about 2 to about 600, or from about 2 to about 500, or from about 2 to about 400, or from about 2 to about 300, or from about 2 to about 200, or from about 2 to about 100, or from about 2 to about 90, or from about 2 to about 80, or from about 2 to about 70, or from about 2 to about 60, or from about 2 to about 50, or from about 2 to about 40, or from about 2 to about 30, or from about 2 to about 20, or from about 2 to about 10, or from about 3 to about 10, or from about 2 to about 5, or from about 2 to about 3 layers of interconnected polymer filaments. In still further embodiments, the device comprises about or at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or 10,000 layers of interconnected polymer filaments. In further embodiments, the device comprises 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or 10,000 layers of interconnected polymer filaments.

Devices disclosed herein can be produced to achieve high permeability. For example and without limitation, in various embodiments a device of the disclosure is permeable to a molecule such as bovine serum albumin (BSA) such that the movement of the BSA through the multilayered structure to the lipid bilayer surface is nearly instantaneous. In addition or in the alternative, devices of the disclosure can exhibit ion selective permeability by producing layers of polymer filaments that alternate in charge polarity.

In some embodiments, each of the at least two layers of interconnected polymer filaments comprises a polypeptide. In further embodiments, the polypeptide is a cytoskeletal polypeptide. In some embodiments, the cytoskeletal polypeptide is a catenin, an intermediate filament protein, a microfilament protein, or a microtubule protein.

As used herein a “polypeptide” refers to a polymer comprised of amino acid residues. In some aspects of the disclosure, a device is linked to an external polypeptide as described herein. Polypeptides are understood in the art and include without limitation an antibody, an enzyme, a structural polypeptide and a hormone.

Polypeptides of the present disclosure may be either naturally occurring or non-naturally occurring. Naturally occurring polypeptides include without limitation biologically active polypeptides (including antibodies) that exist in nature or can be produced in a form that is found in nature by, for example, chemical synthesis or recombinant expression techniques. Naturally occurring polypeptides also include lipoproteins and post-translationally modified proteins, such as, for example and without limitation, glycosylated proteins. Non-naturally occurring polypeptides contemplated by the present disclosure include but are not limited to synthetic polypeptides, as well as fragments, analogs and variants of naturally occurring or non-naturally occurring polypeptides as defined herein. Non-naturally occurring polypeptides also include proteins or protein substances that have D-amino acids, modified, derivatized, or non-naturally occurring amino acids in the D- or L-configuration and/or peptidomimetic units as part of their structure. The term “protein” typically refers to large polypeptides. The term “peptide” generally refers to short (e.g., about 50 amino acids or less) polypeptides.

Non-naturally occurring polypeptides are prepared, for example, using an automated polypeptide synthesizer or, alternatively, using recombinant expression techniques using a modified oligonucleotide which encodes the desired polypeptide.

In some embodiments, the polypeptide is a cytoskeletal protein. See, e.g., Alberts, Johnson, Lewis, Morgan, Raff, Roberts, Walter, Wilson, Hunt, Molecular Biology of the Cell, Ch. 16, 6th ed., Garland, 2015. In further embodiments, the cytoskeletal protein is an actin filament, catenin, an intermediate filament protein, a microfilament protein, or a microtubule protein. In some embodiments, the catenin is alpha catenin, beta catenin, or gamma catenin. In further embodiments, the intermediate filament protein is desmin, glial fibrillary acidic protein, keratin, nestin, or vimentin. In some embodiments, the microfilament protein is actin, actinin, filamin, gelsolin, myosin, profilin, tensin, tropomyosin, troponin, or a derivative thereof. In some embodiments, the microtubule protein is dynein, tubulin, or kinesin. In living cells, the formation and destruction of actin filaments (F-actin) is a continuously dynamic process that is driven by a host of molecular interactions [Hill et al., International review of cytology 1982, 78, 1-125]. Once stable filaments are formed, they can be anchored to the membrane by spectrin [Hartwig et al., Protein Profile 1994, 1, 706-778]. The unique interlocking structure of actin monomers forms filaments in a webbing that can propel shape changes in the plasma membrane. Yet, the actin web maintains a thickness that approximates a molecularly thin two-dimensional sheet with large openings. This system uniquely preserves several important membrane properties such as lipid fluidity, direct diffusional access to solution, and the high electrical resistance necessary for single-nanopore sensing applications.

In any of the aspects and embodiments of the disclosure, a multiple-layer device is contemplated. Controlled multiple-layering is contemplated for all filamentous networks comprising, e.g., proteins, oligonucleotides, polymer gels, hydrogels, and a combination thereof. Use of cytoskeletal proteins in devices described herein is important for constructing multiple networked layers. Multiple layers allow for the extension of the 2D-form into a 3D-form for the device. In some embodiments, a 3D network provides further increased resistance to mechanical stress as well as protection from membrane dehydration. The properties of 3D networkability translate into commercially significant features such as extended shelf-life of the device, mechanical durability, field-worthiness, transportability, and reusability.

As used herein a “fragment” of a polypeptide is meant to refer to any portion of a polypeptide or protein smaller than the full-length polypeptide or protein expression product.

As used herein an “analog” refers to any of two or more polypeptides substantially similar in structure and having the same biological activity, but can have varying degrees of activity, to either the entire molecule, or to a fragment thereof. Analogs differ in the composition of their amino acid sequences based on one or more mutations involving substitution, deletion, insertion and/or addition of one or more amino acids for other amino acids. Substitutions can be conservative or non-conservative based on the physico-chemical or functional relatedness of the amino acid that is being replaced and the amino acid replacing it.

As used herein a “variant” refers to a polypeptide, protein or analog thereof that is modified to comprise additional chemical moieties not normally a part of the molecule. Such moieties may modulate, for example and without limitation, the molecule's solubility, absorption, and/or biological half-life. Moieties capable of mediating such effects are disclosed in Remington's Pharmaceutical Sciences (1980). Procedures for coupling such moieties to a molecule are well known in the art. In various aspects, polypeptides are modified by biotinylation, glycosylation, pegylation, and/or polysialylation.

Fusion proteins, including fusion proteins wherein one fusion component is a fragment or a mimetic, are also contemplated. A “mimetic” as used herein means a peptide or protein having a biological activity that is comparable to the protein of which it is a mimetic.

Oligosaccharides useful in the devices and methods disclosed herein include any carbohydrates comprising between about two to about ten monosaccharides or more connected by either an alpha- or beta-glycosidic link. Oligosaccharides are found throughout nature in both the free and bound form.

Oligonucleotides contemplated by the present disclosure include DNA, RNA, modified forms and combinations thereof. The oligonucleotide, in various embodiments, is single stranded or double stranded. Accordingly, in some aspects, a device of the disclosure is linked to an external oligonucleotide that comprises DNA. In some embodiments, the DNA is double stranded, and in further embodiments the DNA is single stranded. In further aspects, a device of the disclosure is linked to an external oligonucleotide that comprises RNA, and in still further aspects a device of the disclosure is linked to an external oligonucleotide that comprises double stranded RNA. The term “RNA” includes duplexes of two separate strands, as well as single stranded structures. Single stranded RNA also includes RNA with secondary structure.

An “oligonucleotide” is understood in the art to comprise individually polymerized nucleotide subunits. The term “nucleotide” or its plural as used herein is interchangeable with modified forms as are known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases adenine (A), guanine (G), cytosine (C), thymine (T) and uracil (U). Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and WO 97/12896, the disclosures of which are incorporated herein by reference. Modified nucleotides include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making oligonucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the oligonucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Oligonucleotides contemplated for use in the devices and methods of the disclosure are from about 5 nucleotides to about 1,000,000 nucleotides in length. More specifically, in some embodiments a device of the disclosure is linked to an external oligonucleotide that is about 5 to about 900,000, about 5 to about 800,000, about 5 to about 700,000, about 5 to about 600,000, about 5 to about 500,000, about 5 to about 400,000, about 5 to about 300,000, about 5 to about 200,000, about 5 to about 100,000, about 5 to about 90,000, about 5 to about 80,000, about 5 to about 70,000, about 5 to about 60,000, about 5 to about 50,000, about 5 to about 40,000, about 5 to about 30,000, about 5 to about 20,000, about 5 to about 10,000, about 5 to about 9,000, about 5 to about 8,000, about 5 to about 7,000, about 5 to about 6,000, about 5 to about 5,000, about 5 to about 4,000, about 5 to about 3,000, about 5 to about 2,000, about 5 to about 1,000, about 5 to about 900, about 5 to about 800, about 5 to about 700, about 5 to about 600, about 5 to about 500, about 5 to about 400, about 5 to about 300, about 5 to about 200, about 5 to about 100, about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all oligonucleotides intermediate in length of the sizes specifically disclosed to the extent that the oligonucleotide is able to achieve the desired result. Accordingly, oligonucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 100,000, 200,000, 300,000, 400,000, 500,000, 600,000, 700,000, 800,000, 900,000, 1,000,000 or more nucleotides in length are contemplated. In further embodiments, oligonucleotides of at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 5,500, at least 6,000, at least 6,500, at least 7,000, at least 7,500, at least 8,000, at least 8,500, at least 9,000, at least 9,500, at least 10,000, at least 20,000, at least 30,000, at least 40,000, at least 50,000, at least 60,000, at least 70,000, at least 80,000, at least 90,000, at least 100,000, at least 200,000, at least 300,000, at least 400,000, at least 500,000, at least 600,000, at least 700,000, at least 800,000, at least 900,000, or at least 1,000,000 nucleotides in length are contemplated.

Cross-linking sites. In any of the aspects or embodiments of the disclosure, one or more layers of polymer filaments comprises one or more cross-linking sites. The association of, e.g., one layer of polymer filaments to another layer of polymer filaments and/or the association of one layer of polymer filaments to a lipid bilayer is facilitated through the association of anchors and linker molecules. A cross-linking site comprises two anchors originating from two adjacent layers or originating from a layer of polymer filaments to a lipid bilayer that either link together directly or are bridged by a linker molecule. In any of the aspects or embodiments of the disclosure, an anchor is covalently tethered to either a lipid molecule, or a polymer filament (e.g., an actin monomer), on one end, and a “key” moiety (e.g., biotin) on the other. In any of the aspects or embodiments of the disclosure, the lipid bilayer comprises one or more anchors. In some embodiments, each of the at least two layers of interconnected polymer filaments comprises an anchor. In various embodiments, the at least two layers of interconnected polymer filaments are linked to each other through a cross-linking site. In some embodiments, a linker molecule is used to trigger the linkage between, e.g., an anchor on a layer of polymer filaments and an anchor on a lipid bilayer. In some embodiments, a layer of polymer filaments and a lipid bilayer each comprise one or more anchors that comprise biotin, such that addition of an avidin linker molecule will generate one or more cross-linking sites and cause the linkage of the layer of polymer filaments to the lipid bilayer. Linker molecules contemplated by the disclosure include, but are not limited to, streptavidin, avidin, neutravidin, a biotin binding protein, an antibody directed against digoxygenin, or a combination thereof.

In some embodiments, an anchor directly binds to another anchor in the absence of a linker molecule. Thus, in some embodiments, a linker molecule is not required to join an anchor to another anchor.

In any of the aspects or embodiments of the disclosure, layers in a multilayer structure are connected to one another directly through a combination of electrostatic attractions, ionic bonds, and hydrogen bonds. Accordingly, interlayer connections formed by electrostatic attractions, ionic bonds, and hydrogen bonds add to the interlayer connections created when cross-linking sites are present. The additional forces from this type of direct, layer-to-layer binding add strength to the multilayer structure, and the enhanced strength of the multilayer structure facilitates the use of very thin structures in various applications. By way of example, multilayer structures that are strong and thin form short conduits that can possess large diameters. These short conduits of large diameter greatly enhance diffusive permeability. The concepts of conduits, diameter, and diffusive permeability are described in greater detail herein below.

In addition, in various embodiments, at least one of the at least two layers of interconnected polymer filaments are linked to the lipid bilayer through a cross-linking site. In any of the aspects or embodiments of the disclosure, the cross-linking site comprises (i) biotin and streptavidin; (ii) spectrin; (iii) avidin, neutravidin, or a biotin binding protein; (iv) a bridge protein from the ERM family (e.g., ezrin, radixin, or moesin); (v) a bridge protein from the formin family (e.g., myosin I, integrin, tensin, catenin (alpha-, beta-, or gamma-); (vi) a transmembrane glycoprotein (e.g., CD44); or (vii) digoxygenin and an antibody directed against digoxygenin. The number of anchors that are present on a layer of polymer filaments or a lipid bilayer varies, and affects aspects of the device that are related to permeability. In general, the presence of more anchors will increase filament density, decrease the conduit diameter, and make conduits more highly branched. In turn, the presence of more anchors will decrease the size of the molecule that is permeable. Further, if the number of anchors is increased by depositing more filaments within a layer, and those filaments bear an electrostatic charge, then the increased electric field strength of that layer will alter permeability to certain ions. The number of anchors present on either a layer of polymer filaments or a lipid bilayer can be expressed as a percentage of the layer of polymer filaments or lipid bilayer that includes an anchor. In various embodiments, from about 0.001% to 100% of the available sites on a layer of polymer filaments or a lipid bilayer comprises an anchor. In further embodiments, about or at least about 1%, 5%, 10%, 15%, 20%, 30%, 40%, 50%, 60%, 70%, 80%, or 90% of the available sites on a layer of polymer filaments or a lipid bilayer comprises an anchor. Alternatively, the number of cross-linking sites between layers of polymer filaments or between a lipid bilayer and a layer of polymer filaments can be expressed as number per unit area and, in various embodiments, is from about 0.04 to about 12×10⁴ or from about 10² to about 10³ cross-linking sites per μm².

As described herein, interlayer linkages can be either (1) cross-linking sites that are composed of covalent and strong non-covalent bonds (for example and without limitation, biotin-avidin) or (2) direct interlayer bonds including electrostatic attractions (charge bearing layers of opposite polarity), ionic bonds, and hydrogen bonds.

It is also contemplated herein that individual layers of interconnected polymer filaments utilize different interlayer linkage types. In some embodiments, some layers are strictly strong non-covalent, while others are strictly ionic, or any combination thereof.

Bilayer support. The design of the bilayer support disclosed herein closely mimics actual cytoskeletal structures that have produced success in living cells. A membrane according to the disclosure that is linked to at least two layers of interconnected polymer filaments remains stable for extended periods of time, including indefinitely. Without such linkage to the at least two layers of interconnected polymer filaments, the membrane is much less stable and durable, and quickly loses its structure. Methods of preparing a device of the disclosure are described herein below.

Materials useful in the preparation of the devices of the present disclosure include those that serve as functional groups to create a lipid bilayer. For example and without limitation, the bilayer, in various embodiments, comprises a plurality of lipid functional groups comprising, for example and without limitation, diphytanoyl 1,2,-diacyl-sn-glycero-3-[phosphor-L-serine] (DiPHyPC), 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), or 1,2-dihexadecanoyl-sn-glycero-3-phosphoethanolamine (DPPE). As a category, lipids are a group of natural and synthetic molecules that include fats/fatty acids, waxes, sterols, fat-soluble vitamins (such as vitamins A, D, E, and K), monoglycerides, diglycerides, triglycerides, and phospholipids. Each of these subcategories is contemplated by the disclosure. Thus, the disclosure contemplates any lipid compound may be used. Even more generally, any amphiphilic compound that can be used to create a membrane with hydrophilic and hydrophobic regions is contemplated to be useful in the devices and methods of the disclosure. Derivatized lipids are also contemplated by the disclosure. For example and without limitation, in some embodiments a lipid of the disclosure is biotinylated such that biotin is itself linked to the lipid head group using a carbonaceous tether ranging from a few to more than a dozen atoms.

In some embodiments, the device further comprises at least one ion channel forming at least one pore through the lipid bilayer. Ion channels provide the molecular basis for nerve activity and mediate the selective transport of ions and macromolecules. In addition, some ion channels connect cells together to form large-scale functioning tissue whereas others act as lethal toxins. It has been shown that channels could act as components of sensors to detect a variety of analytes including ions, small molecules, polynucleotides, and polypeptides. According to further embodiments of the disclosure, the device comprises one pore per aperture. In some embodiments, the device comprises a plurality of apertures. In further embodiments, the device comprises from about 1 to about 1,000,000, or from about 1 to about 900,000, or from about 1 to about 800,000, or from 1 to about 700,000, or from about 1 to about 600,000, or from about 1 to about 500,000, or from about 1 to about 400,000, or from 1 to about 300,000, or from about 1 to about 200,000, or from about 1 to about 100,000, or from about 1 to about 50,000, or from about 1 to about 500, or from about 1 to about 50, or from about 2 to about 50, or from about 5 to about 50, or from about 10 to about 50, or from about 10 to about 40, or from about 10 to about 30, or from about 1 to about 1000, or from about 1 to about 10,000, or from about 10 to about 100, or from about 20 to about 50 apertures. In various embodiments, the device comprises about or at least about 10, about or at least about 20, about or at least about 30, about or at least about 40, about or at least about 50, about or at least about 60, about or at least about 70, about or at least about 80, about or at least about 90, about or at least about 100, about or at least about 200, about or at least about 500, about or at least about 1,000, about or at least about 2,000, about or at least about 3,000, about or at least about 5,000, about or at least about 7,000, about or at least about 10,000, about or at least about 50,000, about or at least about 100,000, about or at least about 200,000, about or at least about 300,000, about or at least about 400,000, about or at least about 500,000, about or at least about 600,000, about or at least about 700,000, about or at least about 800,000 about or at least about 900,000, about or at least about 1,000,000 or more apertures. In some embodiments, the aperture is from about 10 nanometers (nm) to about 1 millimeter (mm) in diameter. In further embodiments, the aperture is from about 10 nm to about 900 microns (μm), or from about 10 nm to about 800 μm, or from about 10 nm to about 700 μm, or from about 10 nm to about 600 μm, or from about 10 nm to about 500 μm, or from about 10 nm to about 400 μm, or from about 10 nm to about 300 μm, or from about 10 nm to about 200 μm, or from about 10 nm to about 100 μm, or from about 50 μm to about 500 μm, or from about 10 nm to about 1 μm in diameter. In further embodiments, the aperture is from about 10 μm to about 100 μm in diameter. In some embodiments, the aperture is about or at least about 10 nm, about or at least about 20 nm, about or at least about 30 nm, about or at least about 40 nm, about or at least about 50 nm, about or at least about 60 nm, about or at least about 70 nm, about or at least about 80 nm, about or at least about 90 nm, about or at least about 100 nm, about or at least about 200 nm, about or at least about 300 nm, about or at least about 400 nm, about or at least about 500 nm, about or at least about 600 nm, about or at least about 700 nm, about or at least about 800 nm, about or at least about 900 nm, about or at least about 1 μm, about or at least about 100 μm, about or at least about 200 μm, about or at least about 300 μm, about or at least about 400 μm, about or at least about 500 μm, about or at least about 600 μm, about or at least about 700 μm, about or at least about 800 μm, about or at least about 900 μm, or about or at least about 1 mm in diameter.

In further embodiments, a device of the disclosure is positioned over the top of a cavity/microcavity that is etched into a substrate. Substrates contemplated by the disclosure include, without limitation, a polymer resin, glass, or a semiconductor. In some embodiments, the cavity/microcavity is a divot formed in glass by, for example and without limitation, micromachining or lithographic methods. Traditional apertures are understood in the art to comprise a small hole through a thin 2D film that separates two large aqueous-filled chambers. The volume of traditional chambers is on the order of milliliters. However, a device of the disclosure is contemplated to be implemented, in some embodiments, using an aperture created by a micromachined divot that is filled with a tiny amount of water (e.g., 10-1000 nanoliters). This type of aperture, which is defined by the rim of the micromachined divot, is effectively closed to bulk solution on the bottom side, but the top side of the aperture is in contact with a larger volume of water (see, e.g., U.S. Patent Application Publication No. 20150152494, which is incorporated by reference herein in its entirety). Lipid membranes formed over well-type apertures can be more stable than traditional membranes formed on a 2D film partition. Use of a device of the disclosure will further improve stability and performance of membranes on well-type apertures.

In some embodiments, the ion channel forms a pore all the way through the lipid bilayer. In some embodiments, the pore is a protein ion channel. Protein ion channels are naturally occurring proteins or derivatives thereof having a biological function. In some embodiments, the ion channel is produced by bacteria. Suitable protein ion channels include, but are not limited to, Staphylococcus aureus alpha-hemolysin, Bacillus anthracis protective antigen 63, gramicidin, MspA (Mycobacterium smegmatis), OmpF porin, Kapton, OmpG, and ClyA (Salmonella typhimurium), a non-naturally occurring compound, and derivatives thereof. The ion channel may also be a synthetic, or non-naturally occurring compound. Suitable ion channels are disclosed in U.S. Pat. No. 7,504,505, which is incorporated herein by reference in its entirety.

### Methods of Forming and Tailoring a Device of the Disclosure

The disclosure also provides methods of forming and tailoring a device for various applications. In general, creation of a multilayer device of the disclosure follows an iterative (A-Bₙ)_{N} algorithm, where A designates a saturating linker molecule deposition step, B designates polymer filament (e.g., actin) deposition, n represents the number of subsaturation additions of polymer filaments, and N indicates the number of connected layers. In any of the aspects or embodiments of the disclosure, wash steps are included to remove residual unbound material after saturation and follow the addition of linker (A) and polymer filaments (B), respectively. To create multilayers (N), linker molecules and polymer filaments are added iteratively. Linker molecules are added at a concentration high enough to saturate the anchors within the deposited polymer filaments. These steps are repeated to form the desired number of multiple layers (N).

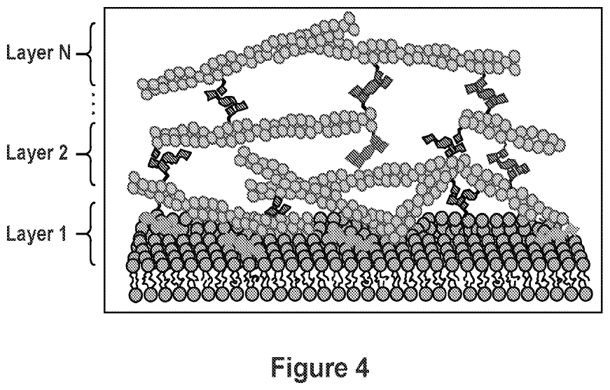

As described herein, a membrane of the disclosure is a composite structure comprising a lipid bilayer that is linked to a substrate. The disclosure further contemplates, in various embodiments, that linkage to the substrate can occur through physical adsorption, or bonded covalent interactions, with lipid or modified lipids and functionalized surface moieties on the substrate (see, e.g., Bright et al., ACS Appl. Mater. Interfaces 2013, 5, 11918-11926 and Bright et al., ACS Biomater. Sci. Eng., 2015, 1, 955-963). The membrane is further linked to at least one of the at least two layers of interconnected polymer filaments as described herein. The at least two layers of interconnected polymer filaments are external to the membrane. An “external” layer of polymer filaments is one that is not part of the lipid bilayer per se, but is later linked to the lipid bilayer through one or more cross-linking sites (see, e.g., FIG. 4) and/or direct non-covalent or covalent interactions (e.g., direct covalent or non-covalent bonding of filament side chains to lipid head groups). Further embodiments comprise a non-protein polymer tether that allows controllable spacing between the membrane and the filamentous network. See, for example, U.S. Pat. No. 7,504,505. In some embodiments, the number of cross-linking sites on the membrane is varied. As described herein, the number of cross-linking sites on the membrane may be expressed as a number per unit area or as a percentage of the membrane that comprises a cross-linking site. In some embodiments, the cross-linking site comprises (i) biotin and streptavidin; (ii) spectrin; (iii) avidin, neutravidin, or a biotin binding protein; (iv) a bridge protein from the ERM family (e.g., ezrin, radixin, or moesin); (v) a bridge protein from the formin family (e.g., myosin I, integrin, tensin, catenin (alpha-, beta-, or gamma-); (vi) a transmembrane glycoprotein (e.g., CD44); or (vii) digoxygenin and an antibody directed against digoxygenin. In general, the protein bridge may comprise any polypeptide and an antibody directed against the polypeptide. The disclosure further contemplates the use of any of several filament stabilizer molecules to further enhance stability and mechanical properties of the filamentous network. Stabilizer molecules bind along the side of a filament and may or may not participate in filament-filament linkages. Examples of actin-stabilizing molecules are tropomyosin and phalloidin. An example of a microtubule-stabilizing molecule is taxol. Further examples of microtubule stabilizer proteins are microtubule-associated proteins (MAPs), and include but are not limited to tau and MAP-2. In further embodiments, the disclosure contemplates the use of filament polymerization enhancers. Polymerization enhancers bind to monomer units to initiate and accelerate filament growth while also enhancing the mechanical properties of the external filamentous network. Examples of polymerization enhancers include but are not limited to XMAP215 (relevant to microtubules), gamma-TuRC (relevant to microtubules), formin (relevant to actin), and profilin (relevant to actin).

In further embodiments, the linkage comprises a bridge-forming molecule that enables networking (crosslinking) inside the at least two layers of interconnected polymer filaments. Such molecules enable filament-filament linkages, as opposed to filament-lipid bilayer linkages. By way of example, molecules such as streptavidin, avidin, or neutravidin can serve two purposes: (i) as a bridge to link the at least two layers of interconnected polymer filaments to a membrane (e.g., a biotinylated membrane); and (ii) as a potential actin-actin crosslinker (i.e., a linker that is inside each of the at least two layers of interconnected polymer filaments). Thus, creating multiple polymer filament (e.g., actin) layers by bridging actin filaments one molecular layer at a time (or using filament-to-filament bridges to increase the 2D density of a sub-molecular layer) is contemplated herein. In further embodiments, a protein from the gelsolin family (e.g., villin) is contemplated for use in forming bridges within the at least two layers of interconnected polymer filaments. Additionally, fascin, fimbrin, alpha-actinin, spectrin, filamin, dystrophin, ARP complex, gamma-TuRC, and filaggrin are actin crosslinkers or binding proteins contemplated for use according to the disclosure. Plectin acts as a linker between all three major categories of cytoskeletal filaments (i.e., actin filaments, microtubules, and intermediate filaments). In still further embodiments, links within the at least two layers of interconnected polymer filaments comprise modified nucleotides, oligosaccharides, proteins, or peptide strands. Any type of chemical linkage used to join individual filaments (e.g., via covalent, non-covalent, or ionic bonds) for molecular level control of the 2D and 3D filament density and branching structure within the at least two layers of interconnected polymer filaments is contemplated by the disclosure.

Like the hydrogel sandwich, the first step in forming a device of the disclosure is to form a black lipid membrane (BLM) that possesses electrical characteristics necessary for single-channel recording. Then multiple layers of polymer filaments (each of approximate single-layer thickness or less) are linked to the lipid bilayer. Polymer filaments that are external to the membrane are discussed further herein, and include, in some embodiments, filamentous actin. In any of the aspects or embodiments of the disclosure, chemical links to the membrane are formed by establishing a cross-linking site to connect the lipid bilayer to the at least two layers of interconnected polymer filaments. The cross-linking site is formed via anchors and linker molecules, each as described herein. Also as described herein, direct layer-to-layer connections (e.g., connections may also be established via electrostatic forces, ionic bonds, and hydrogen bonds). In some embodiments, the cross-linking site comprises biotin (anchor) and avidin (a linker molecule) which creates a strong integral connection to the hydrophobic interior. Accordingly, in some aspects, the disclosure provides a method of forming a device comprising (a) providing a lipid bilayer, the lipid bilayer comprising a first anchor, wherein the lipid bilayer is associated with a substrate, the substrate comprising an aperture and an electrode; (b) applying a linker molecule; (c) providing a first layer of polymer filaments comprising a second anchor, thereby creating a cross-linking site between the first anchor, the linker molecule, and the second anchor, thereby linking the lipid bilayer to the first layer of polymer filaments; (d) applying the linker molecule; (e) providing a second layer of polymer filaments comprising a third anchor, thereby creating a cross-linking site between the second anchor, the linker molecule, and the third anchor, thereby linking the first layer of polymer filaments to the second layer of polymer filaments; and (f) inserting a pore into the lipid bilayer, thereby forming the device. In some embodiments, the method further comprises applying the linker molecule between steps (e) and (f); and providing a third layer of polymer filaments comprising a fourth anchor, thereby creating a cross-linking site between the third anchor, the linker molecule, and the fourth anchor, thereby linking the second layer of polymer filaments to the third layer of polymer filaments. This process of adding successive layers may be continued in a similar fashion to achieve a network of interconnected polymer filaments with a desired number of layers. Each layer of the network of interconnected polymer filaments may be tailored with respect to, for example and without limitation, density, number of cross-linking sites, thickness, strength, electrical resistivity and composition, each of which is described in more detail herein.

In some embodiments, the electrode is required to sense the translocation of anlaytes via electrical measurement techniques. In further embodiments, an optical method for sensing the translocation of an analyte is utilized that does not require and electrode.

The disclosure also provides methods of tuning or tailoring a device as disclosed herein to, for example, adjust the permeability of the device to fit a desired application. Such methods include adjusting the density of one or more layers of interconnected polymer filaments, adjusting the charge polarity of one or more layers of interconnected polymer filaments, adjusting the charge density of one or more layers, or a combination thereof.

As described herein, the density of each layer of interconnected polymer filaments may be adjusted, and adjusting the density of a layer will result in a change in the size of the conduit between interconnected polymer filaments. In some embodiments, each of the at least two layers comprises a conduit between the interconnected polymer filaments. In general, a layer that possesses a higher density of polymer filaments will have a smaller and more highly branched conduit and will therefore be permeable only to smaller molecules. Conversely, a layer that possesses a lower density of polymer filaments will have a larger and less highly branched conduit and will therefore be permeable to larger molecules. In various embodiments, the conduit is from about 10⁻³ to about 10⁰ microns (μm) in diameter. In further embodiments, the conduit is, is about, or is at least about 0.001, 0.005, 0.007, 0.01, 0.05, 0.07, 0.09, 0.1, 0.2, 0.3, 0.4, 0.5, 0.6, 0.7, 0.8, 0.9, or 1 μm in diameter.

In some embodiments, each layer of interconnected polymer filaments or linker molecules has an associated charge. The extent of the charge of each layer is based on the isoelectric point of the material used in each layer (for example and without limitation, F-actin, avidin, etc.) and the relative pH and ionic strength of the buffer. The surface charge density of a particular layer can be adjusted, and is dependent upon the isoelectric point (pI) of the material chosen for each layer. By way of example, if a layering pattern of A-B-A-B-A-B (N=3 layers) is utilized, where A is a linker molecule and B is a polymer filament, and where A has a high pI and B has a low pI, then the excess charge on each layer is defined by the material's pI relative to the pH. At pH=7, for example, layer A (e.g., avidin) possesses positive charge and layer B (e.g., actin) possesses negative charge. Therefore, at pH=7 both ionic and electrostatic cross-links are formed between A and B. Furthermore, this charge creates ion-selective permeability. Ion selective permeability refers to the ability to allow certain ions through but not others. At high ionic strengths, the amount of charge on both layers is reduced/neutralized via ion pairing, leaving only the cross-linking sites (comprising covalent and strong non-covalent bonds) to hold layers together. At low ionic strength, all layers that have pIs that differ from the pH will bear a charge (i.e., positive charge for pH<pI and negative charge for pH>pI) and both electrostatic, ionic, and cross-linking sites hold layers together. In some embodiments, layers having alternating charge polarity are contemplated for use in a device to add strength to the device.

In a specific example for illustration, a layering pattern such as A-B-C-B-D-B (N=3 layers) may be employed, where A, C, D layers comprise linker molecules with different pIs. Avidin (A) has a pI=10. Neutravidin (C) has a pI=6.3, and streptavidin (D) has a pI=5.0. Thus, at a pH of 7, each layer (A, C, D) will have a distinct charge polarity and magnitude. And the amount of charge can be effectively reduced through ion pairing that occurs when the ionic strength is high.

Formation success frequency. Methods of the disclosure produce devices with high formation success frequency. Formation success frequency is a measure of the percentage of devices that are formed that are free of leaks. Leaks in the device are detected by determining the resistance of the membrane. Structures that produce less than approximately 2 GΩ of resistance are generally considered unsuccessful. Methods provided herein routinely result in formation of structures having resistances ranging from about 10-100 GΩ. In various embodiments, methods of the disclosure produce devices having a formation success frequency that is from about 70% to about 90% or more. In further embodiments, methods of the disclosure produce devices having a formation success frequency that is, is about, or is at least about 70%, 75%, 80%, 85%, 90%, 95%, or 99%.

In some aspects, the disclosure provides a method of forming a device comprising: (a) providing a lipid bilayer comprising biotin, wherein the lipid bilayer is associated with a substrate, the substrate comprising an aperture and an electrode; (b) applying avidin; (c) providing a first layer of polymer filaments comprising biotin, thereby creating a cross-linking site between the biotin on the lipid bilayer, the avidin, and the biotin on the first layer of polymer filaments, thereby linking the lipid bilayer to the first layer of polymer filaments; (d) applying avidin; (e) providing a second layer of polymer filaments comprising biotin, thereby creating a cross-linking site between the biotin on the first layer of polymer filaments, the avidin, and the biotin on the second layer of polymer filaments, thereby linking the first layer of polymer filaments to the second layer of polymer filaments; and (f) inserting a pore into the lipid bilayer, thereby forming the device. In some embodiments, the method further comprises applying avidin between steps (e) and (f), and providing a third layer of polymer filaments comprising biotin, thereby creating a cross-linking site between the biotin on the second layer of polymer filaments, the avidin, and the biotin on the third layer of polymer filaments, thereby linking the second layer of polymer filaments to the third layer of polymer filaments.

### Methods of Using a Device of the Disclosure

Various methods for using the devices of the disclosure are contemplated. In some aspects, methods of analyzing a target polymer are provided comprising contacting the target polymer to a device of the disclosure, allowing the target polymer to move with respect to the at least one pore present in the device to produce a signal, and monitoring the signal corresponding to the movement of the target polymer with respect to the pore, thereby analyzing the target polymer. In some embodiments, the signal monitoring comprises measuring a monomer-dependent characteristic of the target polymer while the target polymer moves with respect to the pore. In further embodiments, the monomer dependent property is the identity of a monomer or the number of monomers in the polymer. The target polymer, in various embodiments, is an oligonucleotide, a peptide, or a polypeptide, each as described herein. In some embodiments, the analyzing comprises a chemical characterization. In further embodiments, the chemical characterization is a characterization of DNA, a synthetic polymer, a small molecule, or an ion. In some embodiments, the characterization of DNA comprises nucleotide sequencing or genotyping.

Thus, in some embodiments the entire structure including the membrane, aperture, and an ion channel are useful for analysis of polymers. In some embodiments, the analysis comprises DNA and other polynucleotide sequencing. An electrolyte solution containing the DNA is placed on one side of the membrane. Electrolyte is also placed on the other side of the membrane. A voltage is applied through the electrolytes and across the membrane. This causes a DNA strand to gradually pass through the membrane. As the strand passes through, the current passing through the membrane is measured. The current is affected by the number and identity of the nucleotides presently in the pore. When using protein ion channels, there is typically more than one nucleotide in the pore. The identity of each nucleotide is determined from several current measurements as the nucleotide passes through the pore. A synthetic pore may be short enough to hold only one nucleotide. This simplifies the sequencing, as each nucleotide identification is determined from a single current measurement.

In some embodiments, the device further comprises a molecular motor. The molecular motor, in some embodiments, is adjacent to a pore of the device, and the molecular motor is capable of moving a target polymer with respect to the pore. By way of example, a target polymer can be passed through a molecular motor tethered to the surface of a device or embedded in a device, thereby bringing units of the target polymer sequentially to a specific location, preferably in interactive proximity to an agent. Agents contemplated herein include but are not limited to electromagnetic radiation, a quenching source and a fluorescence excitation source. Individual units of the target polymer interact with the agent to produce a detectable signal, and the signals resulting from said interaction are sequentially detected to analyze the polymer. According to some embodiments of the disclosure, individual units of the target polymer are labeled with a fluorophore.

A molecular motor is a compound such as polymerase, helicase, or myosin which interacts with the polymer and is transported along the length of the polymer past each unit. In further embodiments, the molecular motor comprises a DNA polymerase, a RNA polymerase, a ribosome, or an exonuclease. In still further embodiments, the DNA polymerase is selected from E. coli DNA polymerase I, E. coli DNA polymerase I Large Fragment (Klenow fragment), phage T7 DNA polymerase, Phi-29 DNA polymerase, Thermus aquaticus (Taq) DNA polymerase, Thermus flavus (Tfl) DNA polymerase, Thermus Thermophilus (Tth) DNA polymerase, Thermococcus litoralis (Tli) DNA polymerase, Pyrococcus furiosus (Pfu) DNA polymerase, Bacillus stearothermophilus (Bst) DNA polymerase, AMV reverse transcriptase, MMLV reverse transcriptase, and HIV-1 reverse transcriptase.

In some embodiments, the RNA polymerase is selected from T7 RNA polymerase, T3 RNA polymerase, SP6 RNA polymerase, and E. coli RNA polymerase. In some embodiments, the exonuclease is selected from exonuclease Lambda, T7 Exonuclease, Exo III, RecJ₁ Exonuclease, Exo I, and Exo T. In some embodiments, the helicase is selected from E. coli bacteriophage T7 gp4 and T4 gp41 gene proteins, E. coli protein DnaB, E. coli protein RuvB, and E. coli protein rho.

In some embodiments, methods of analyzing a target polymer further comprise altering the rate of movement of the polymer before, during, or after the signal monitoring. In various embodiments, the alteration of the rate of movement of the polymer is facilitated by the multilayer structure that is added to the lipid bilayer according to the methods provided herein.

The Examples that follow demonstrate the creation of mechanically stabilized bilayers on an array with multiple cross-linked layers of F-actin that are chemically linked to lipid headgroups in the bilayer. Importantly, the layered structure remains highly permeable, allowing open penetration to the lipid bilayer, while maintaining a tight electrical seal (i.e., 10-100 GΩ). The array substrates contain four microcavities machined into the surface of a small fluidic chamber. Enhanced biomembranes were formed over the top of each water-filled microcavity. The bottom of each cavity contains an electrode deposit that permits individual application of a transmembrane voltage and allows measurement of electrical currents across each biomembrane with exquisite sensitivity, down to the level of single ion channels and nanopores. Such sensitivity is ideal for sensing applications.

Other investigators have previously explored synthetic polymer slab gels to strengthen lipid bilayers.³²⁻³⁴ However, the thickness of these supports (>10⁰ μm) significantly reduces the rate of diffusive migration to the bilayer (by approximately 70%), 35 making nanopore sensing difficult. Interestingly, slab hydrogels also do not maximize resistance to mechanical strain.^{34, 36} Numerous other support structures have been explored that are not conjugated to the bilayer,³⁷⁻³⁹ lack a reliable formation success rate, require restrictive lipid headgroup charge criteria, and possess non-ideal resistivity.³⁸ Additionally, in comparison to the approach explored herein, no previous technique possess the inherent capacity for molecular-level layering and design.

Apart from external structures that are added to support the bilayer, lipid molecules within the bilayer have been the target of polymerization experiments.⁴⁰ Although a lipid polymer network allows open access to nanopores embedded in membrane, the polymerization process can result in an electrically leaky partition that is not able to achieve the desirable 10-100 GΩ seal.⁴⁰ High electrical resistance must be maintained across the lipid bilayer in order for nanopore sensing to be optimally successful.

It was previously shown that minimalistic F-actin support structures can enhance the mechanical strength and elastic properties of black lipid membranes (BLMs) formed over an open aperture.³⁶ The layers are molecularly thin, provide a large strain resistance per layer, and allow the formation of a tight electrical seal. In the present disclosure, this idea was extended to construct stronger multilayered and cross-linked structures over an array of microcavities. The experiments described below demonstrate the construction and characterization of such structures. The polymeric support structures that span the opening of each microcavity is referred to as a multilayer structure.^{41, 42} In addition, some embodiments of the disclosure contemplate use of an array chip that is appropriate for nanopore sensing and could be employed for commercial applications directly, or in an expanded form with automated microfluidics.

As described and exemplified herein, cross-linked multilayered structures permit rapid diffusive transport to the bilayer. Thus, multilayered structures enhance the mechanical strength of the lipid bilayer while maintaining the necessary permeability and the high resistivity typical of BLMs. The bilayers are also stable and have a high formation success rate. Moreover, the particular materials chosen to construct multilayered structures are contemplated to resist mechanical, chemical, and electrical stress by employing tailored combinations of thickness, porosity, and electrostatic charge, all within the configuration. The ability to conduct layer-by-layer rational design tailored to specific nanopore sensing applications further distinguishes multilayered structures as disclosed herein from other approaches investigated for bilayer support.

All documents cited herein are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

### EXAMPLES

The following experiments describe the multilayered structure formation process in the context of a bilayer array on a microchip, as well as glass supported bilayers. Permeability tests demonstrated that molecular access to the bilayer is not hindered by the presence of the multilayered structure. Permeability was achieved by controlling the openness of the F-actin network, which can be tailored using the density of linkage sites, the electrostatic interactions between linkers and filaments, and specific F-actin growth and deposition techniques. Interestingly, the conductivity of nanopores embedded in the bilayer suggests that multilayered structures designed with alternating layers of charge can create ion-selective permeability. Results from single-molecule optical measurements that characterize the multilayered structure, its permeability, and the approximate conduit size of the layered network are also presented.

The bilayers described in the following experiments were found to be stable over the duration of the tests (hours-days) and have a formation success rate of approximately 80-90%.

### Example 1

### Materials and Methods

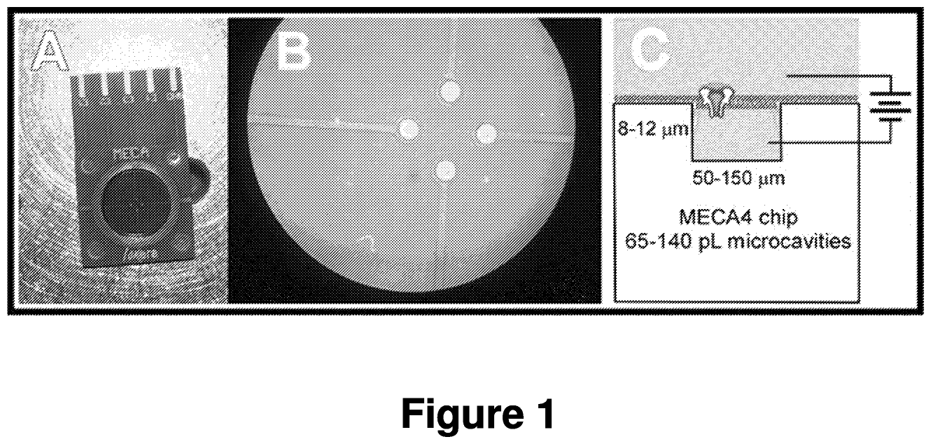

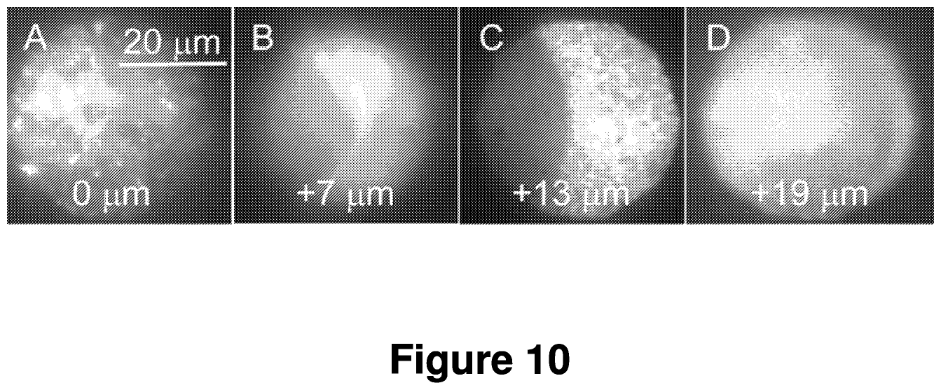

Microarray Chip (MECA4-Fluo). MECA4-Fluo chips are manufactured by lonera and were purchased through Nanion. The chip consists of an approximately 300 μL water-filled chamber that contains an array of four separate microcavities machined into the bottom surface. Each microcavity is approximately 8-12 μm deep with a diameter that ranges between 50-150 μm and holds 65-140 pL of solution. The bottom surface of the microcavity features a small Ag/AgCl electrode that is coupled through printed circuit connections to one of four exposed contacts located at the top of the chip. A ground electrode ring is deposited around the perimeter of the chamber and is coupled to a fifth contact exposed at the top of the chip. These connection points permit independent trans-bilayer voltage control and current monitoring in each microcavity of the array. Lipid bilayers are formed over the top of each cavity. FIGS. 1A and 1B shows an image of the MECA4-Fluo chip and a magnified view of the microcavities. FIG. 10 illustrates a single nanopore puncturing a lipid bilayer that is suspended over the top of a water-filled microcavity.

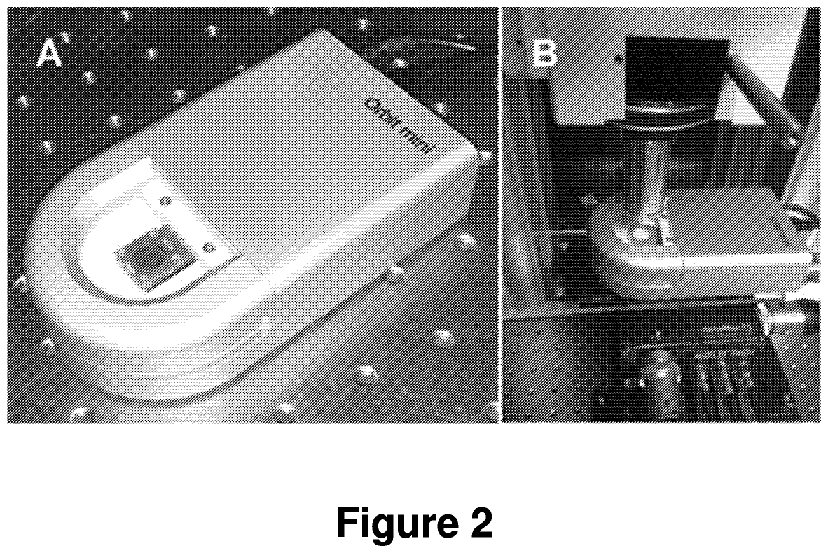

Electrical Measurements in the Orbit Mini. An Orbit Mini was purchased from Nanion to record voltage and current data from the MECA4-Fluo chip. All amplification and filtering electronics are contained within a compact field-worthy unit that connects to a laptop computer via the USB port (FIG. 2). The Orbit Mini electronics enable parallel electrical recordings from each of the four microcavities individually. Both membrane capacitance and current levels were recorded. The specific capacitance of properly formed bilayers was approximately 0.7 pF/μm². A systematic drop in specific capacitance (5-50%) was noted upon addition of a multilayered structure of at least three layers. The Orbit Mini was used in conjunction with an upright fluorescent microscope (FIG. 2B) and high optical magnification (60× objective) to acquire actin filament images once the multilayer-coated bilayers were formed. This configuration allowed optical interrogation of one of the four MECA4 microcavities at a time.

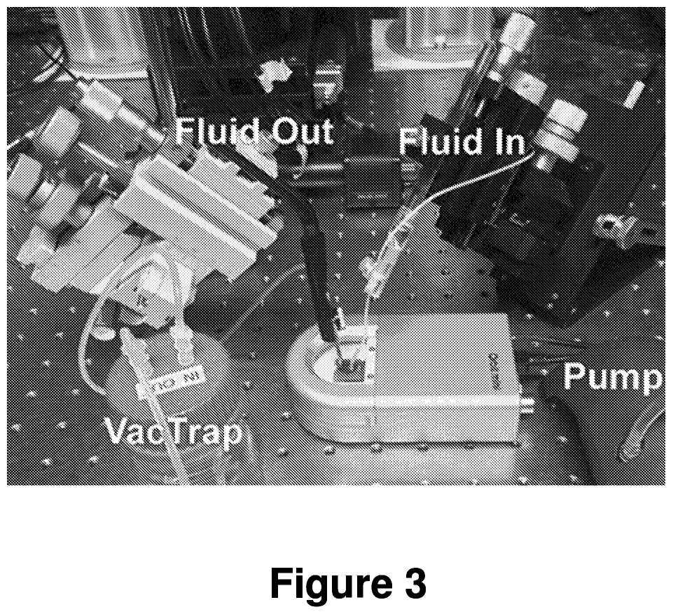

Perfusion Apparatus for Rapid Chamber Washing. In order to create the multilayered polymeric support structures for ruggedizing the lipid bilayer, a manually controlled perfusion system was assembled to enable fast washing of the chamber contents. Rapid solution exchange is necessary for proper deposition and layering of materials on top of the bilayer. To accomplish this, the perfusion system was mounted on two 3D micropositioners in close proximity to the Orbit Mini with a MECA4-Fluo chip. The perfusion system (FIG. 3) allowed aqueous solutions to be exchanged within the MECA4-Fluo chamber at the rate of 1-10 chamber volumes per minute. As typical of most perfusion systems, fluid is delivered via gravity and removed with mild suction.

Simultaneous Optical and Electrical Measurements. By mounting the Orbit Mini to an upright fluorescence microscope, individual mircocavities in the array can be probed for the presence of fluorescent filaments while also being electrically interrogated. A 532 nm laser focused through a 60×, NA 1.0 objective at an intensity ranging from 30-500 W/cm² was used to illuminate the sample. Light emitted from the sample was collected by the objective and sent through a 550 nm dichroic and 560-620 nm bandpass filter before being directed to a Hammamatsu ORCA-05G digital camera. The illuminated area in the focal plane of the objective (approximately 100 μm diam.) does not fill the camera chip in the conjugate image plane. Under these conditions, exposure times of approximately 0.1 second produced high signal-to-noise images of F-actin bound to the bilayer surface. Electrical measurements on the array of bilayers are performed as described above.

Lipid Preparation. To form bilayers that can be chemically linked to the polymeric support structure, 1,2-diphytnoyl-sn-glycero-3-phosphocholine (DiPhyPC) and 1,2-distearoyl-sn-glycero-3-phosphoethanolamine-N-[biotinyl(polyethylene glycol)-2000] (DSPE-PEG(2000) Biotin) were purchased from Avanti Polar Lipids. A mixture of the two lipids was prepared in chloroform and dried under vacuum so that the mole ratio of DSPE-PEG(2000) Biotin ranged from 0.1-1%. The most common concentration employed was 0.1%. Upon bilayer formation, biotin creates a reactive site for attachment with the F-actin polymeric structure and establishes the initial concentration dependent filament packing density. Solutions of the DiPhyPC/DSPE(2000) PEG Biotin were prepared in n-decane and pentane for use in planar lipid bilayer formation at a concentration of 50 mg/mL and 10 mg/mL, respectively.

Actin Filament Preparation. Rabbit skeletal actin monomers >99% pure (Cat: AKL99), biotinylated actin monomers (AB07), and rhodamine-labeled actin monomers (AR05) were ordered form Cytoskeleton, Inc, and mixed to form solutions with mole ratios of 4:1:1, respectively. The lyophilized monomers were resuspended and diluted to 2 μM in a buffer of 0.2 mM CaCl₂) and 5 mM Tris-HCl pH 8.0. The actin solutions were left on ice for an hour to promote depolymerization of preexisting nucleation centers. The remaining nucleation centers were removed by centrifuging for 30 minutes at 4° C. and 16873×g and using the top 80% of the supernatant. To initiate polymerization, concentrated polymerization buffer was added to obtain a final polymerization condition of 10 mM Tris HCl, 2 mM MgCl₂, 50 mM KCl, 1 mM ATP, and 5 mM guanidine carbonate pH 7.5. To stabilize the filaments that were formed and lower the critical concentration required for polymerization, phalloidin was added to the actin monomers at the start of polymerization in a 10-fold molar excess. The actin filaments were left at room temperature for 1 hour to polymerize and then stored at 4° C. until needed.

Linker Preparation. Avidin (Sigma-Aldrich) was commercially purchased and stock solutions were prepared in purified water at 1 mg/mL. Neutravidin was purchased from Thermo Fisher and prepared in an identical fashion. Avidin and neutravidin have four biotin binding sites per molecule and isoelectric points (pI) of 10.5 and 6.3, respectively. Electrostatic fields generated by the linkers at pH=7.5 impact the layering dynamics and the resulting 3D structures of the F-actin multilayers. Solutions of linker were prepared by dilution of the stock in 10 mM Tris HCl, pH 7.5 to concentrations ranging from 0.001-0.01 mg/mL. The concentration chosen was dependent upon biotin concentration with the intension of saturating all available biotin binding sites. Typically 604 of the chosen linker solution was added to the 300 μL chamber and incubated for 10 minutes before washing the chamber with approximately 10 mL of buffer.

Multilayer Structure Formation (Actin Filament Deposition and Layering). To form a multilayer structure, microcavities were first pre-conditioned with lipid in pentane by spreading a small droplet (approximately 0.5 uL, 0.5 mg/mL) over the array. The solution was allowed to dry before adding aqueous buffer (10 mM Tris-HCl, pH=7.5) containing a high concentration of salt (1 M KCl). Using a Teflon brush that had been briefly dipped in the n-decane/lipid solution, the top of each microcavity was gently stroked until bilayers spontaneously formed, as indicated by a large change in the current flowing between the two electrodes. These bilayers were then intentionally ruptured by application of a large DC “zapping” potential. The formation and rupture process was repeated several times to ensure solidified residue completely clears the microcavity and that the n-decane solvent evenly coated the rim of the microcavity. Even solvent distribution promotes formation of a proper annulus and a stable bilayer. Once satisfactory membranes were formed, the solution in the top chamber was exchanged by perfusion to a low-salt buffer (50 mM KCl, 10 mM Tris-HCl, pH=7.5) and the multilayering process began.

Multilayer structure creation follows an iterative (A-wash-Bₙ-wash)_{N} algorithm, where A designates the deposition of a linker (e.g., avidin) to saturate all available biotin binding sites, B designates F-actin deposition, n denotes the number incremental additions of F-actin (to achieve approximate saturation), and N indicates the number of cross-linked layers. Wash steps to remove residual unbound material after saturation follow the addition of the linker (A) and F-actin (B r), respectively. FIG. 4 illustrates layers of F-actin that are cross-linked using biotin-avidin-biotin bridges.

To create the first actin layer, a saturating level of linker was added to the chamber to bind available biotinylated lipids that are incorporated into the bilayer (typically 0.1-1 mole %). Linker concentrations in the deposition chamber were approximately 0.0002-0.002 mg/ml (30-400 nM) and were created by injecting approximately 60 μL of a more concentrated stock solution. This solution was allowed to incubate for 10 minutes. Given the pH of the buffer and the pI of avidin, a positively charged surface was formed. Neutravidin maintains a neutral to slightly negative surface. Washes consisted of 5-10× chamber volume exchanges.

Depending on the desired F-actin filament density, aliquots of pre-polymerized actin are diluted 4-40-fold to reach equivalent g-actin monomer concentrations of 0.05 μM or 0.5 μm. This dilution enables the use of a larger volume when transferring filaments to the bilayer and promotes an even distribution of the filaments in solution. In 30 μl aliquots, the actin filaments are injected into the chamber, creating the final concentration of actin of either 0.01 μM or 0.1 μM. Truncated pipette tips are used whenever transferring polymerized actin in order to minimized damage due to fluidic shear stress. Linker filament injections incubate for up to 10 minutes to enable surface adherence by kinetic sedimentation (neutravidin) or electrostatic attraction (avidin). For many measurements, multiple linker additions (n=3-5) are performed until the apparent surface density of filaments no longer increases. The first layer is completed by rinsing away any excess unbound filaments by perfusion.

Construction of the next layer began by adding avidin at a concentration required for saturating the biotinylated monomers within the deposited actin filaments. After washing away excess avidin by perfusion, F-actin was again added in multiple increments (n=2-5). These steps were then iteratively repeated N times until the desired number of multilayers (A-B) were formed. The final step involved washing the chamber with a high salt buffer (10 mM Tris-HCl, pH=7.5, 1 M KCl) to match the salt concentration originally loaded in the microcavities.

In principle, layering can continue indefinitely, in a manner that is limited only by the time needed for deposition. Enhanced automation may be used, but is not required, for layers of N>3.

Multilayer Structure Permeability Tests on MECA4-Fluo chips. In order for multilayered structures to be useful in nanopore sensing applications, molecules must be able to permeate through the support structure. Permeation was assessed by observing nanopore insertion events in the lipid bilayer that can only arise by diffusion through the multilayer support. Single ion-channel insertion current measurements were conducted using the Orbit Mini by adding alpha-hemolysin (αHL) to the chip chamber after construction of a multilayer structure (N=3). The αHL was purchased from Sigma-Aldrich. Typically, 5-10 μL of 2 μg/mL monomeric protein solution was added to the approximately 300 μL chip chamber volume. The transmembrane voltage was set to 50 mV and conductance measurements were performed in 1M KCl buffer. Voltage and current recordings were logged using EDR4 software. Time recordings were acquired at a sampling rate of 5 kHz with low-pass filtering set to the Nyquist frequency. Clampfit 10.4.1.10 software was used to analyze ion channel current recordings.

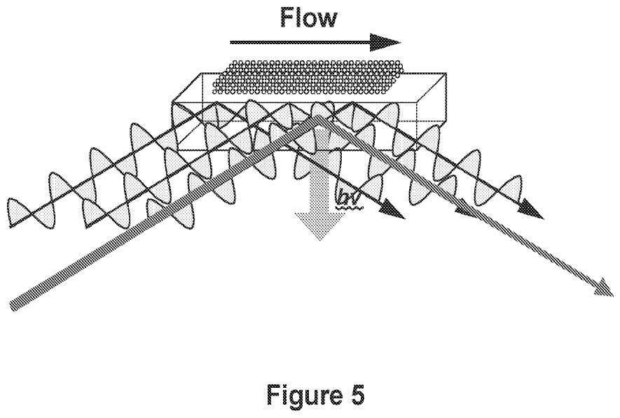

Multilayer Structure Conduit Sizing. In order to characterize the conduit size of multilayered structures, total internal reflection (TIRF) microscopy was employed to probe lipid bilayers formed on glass that are positioned in a fluid flow cell (FIG. 5). This approach served as a proxy for optimizing procedures employed on the MECA4-Fluo array chips. Conduit size was estimated from the density of filaments observed in images from both single layers and multilayers. Due to the overlapping filaments and the diffraction-limited optics, the conduit size estimated for single layers was less than that estimated for multilayers. However, the 3D channel openings that connect bulk solution to the bilayer are most closely characterized by the inter-filament spacing observed in single saturated layers.

TIRF Flow Cell Sample Chamber Preparation. To prepare for TIRF imaging, cover slips were sealed with vacuum grease in a perfusion chamber mounted on an inverted microscope stage. Images were collected on a Nikon Eclipse Ti microscope equipped with a fiber optic coupled TIRF illuminator. The illuminator allowed the laser angle through the objective to be controlled by a micrometer that transitions the laser between TIRF and widefield illumination. Although all of the imaging data of glass supported bilayers were acquired in the TIRF mode, widefield illumination was regularly used to verify the presence of free actin filaments in solution above the bilayer. The illuminator was connected to a single-mode fiber optic that delivered the output from a 532-nm laser. The power output through the objective (Nikon 100×/NA 1.49 oil immersion) in widefield illumination was approximately 5.5 mW. Light arising from the sample was passed through a TRITC filter cube (Chroma). An Andor EMCCD camera (iXon) was used to acquire images on a workstation running Nikon Elements software.

Small unilamellar vesicle (SUV) and Supported Bilayer Preparation. Supported lipid bilayers were formed on borosilicate glass coverslips. The coverslips (15 mm round, No. 1, Warner Instruments) were cleaned by sonicating for an hour in sodium dodecyl sulfate (about 1 g SDS per 300 ml ultrapure water) and sonicating for another hour in reagent grade isopropanol. The cleaned slides were then rinsed and stored in ultrapure water (18.2 MO) until use. Immediately before use, the coverslips were dried using the flame tip from a Bunsen burner. The flow cell chamber in which the glass coverslip was mounted (RC-25F, Warner Instruments) produced laminar flow over the surface by perfusion in a volume of approximately 1 mL.

Small unilamellar lipid vesicles (SUVs) were prepared following standard protocols. Briefly, 1,2-dioleoyl-sn-glycero-3-phosphate (DOPC), 1,2-Distearoyl-sn-Glycero-3-Phosphoethanolamine-N-[Biotinyl(Polyethylene Glycol)2000] (ammonium salt) (DSPE-PEG(2000) biotin) were purchased from Avanti Polar Lipids (Alabaster, AL). N-(Tetramethylrhodamine-6-thiocarbamoyl)-1,2-dihexadecanoyl-sn-glycero-3-phosphoethanolamine, triethylammonium salt (TRITC-DHPE) was purchased from Biotium. All lipids were dissolved in chloroform and mixed to form solutions of 0.1% DSPE-PEG(2000) biotin, or TRITC-DHPE.

Lipid solutions were dried under nitrogen to evaporate the chloroform (approximately 20 minutes for a 1 ml solution). While drying, the vials were held at a 45° angle and gently rotated to facilitate a uniform deposition of lipids over a large surface area within the vial. The lipids were further dried under vacuum for one hour. The dried lipids were resuspended by vortexing in a 10 mM Tris-HCl buffer, pH 7.5, at a lipid concentration of 2.5 mg/ml. The resulting suspensions were sonicated to clarity (15-30 minutes), indicating small unilamellar vesicles (SUVs) have formed.

Directly before addition to the cleaned cover glass in the perfusion chamber, an aliquot of the lipid solution was diluted to 0.5 mg/ml, and 200 μl was added to cover the glass. The solution was left for fifteen minutes to allow vesicle rupture, fusion, and the formation of a continuous 2D bilayer over the glass surface. Residual lipids and vesicles were rinsed from solution using 10 mM Tris-HCl. (10-15 chamber volume exchanges). The effectiveness of this procedure for creating uniform lipid bilayers on glass was verified via TIRF imaging with fluorescent lipids and fluorescence recovery after photobleaching (FRAP) measurements to verify proper bilayer lateral diffusion.

Formation of Multilayer Structures on Glass Supported Bilayers. After formation of the bilayer on the coverslip, the procedures used to construct the multilayer structure on top of the bilayer followed the same (A-wash-Bₙ-wash)_{N} algorithm employed for forming an array of unsupported multilayer structures on the MECA4-Fluo chip.

Single Molecule Diffusion Imaging. Multilayer structures were constructed on glass supported bilayers using a neutravidin linker. Filaments were then deposited following the (A-wash-Bₙ-wash)_{N} procedure to form an N=3 multilayer. Fluorescently labeled bovine serum albumin (BSA) was purchased from Sigma and stock solutions were prepared in water. A diluted quantity was injected into solution above the multilayer while imaging in the TIRF mode. Single BSA molecules permeated the multilayer and were adherent to the bilayer, where they were observed diffusing laterally with a diffusion constant similar to that of lipids within the bilayer (approximately 1×10⁻⁸ cm²/s). To track molecules moving in the plane of the bilayer, a rapid sequence of images (50 ms per frame at 75 ms intervals) was collected and a modified version of Particle Tracker, a plugin for Image J, that is based on a feature point tracking algorithm was used. In order to count the number of fluorescent BSA particles per unit area, image sequences were first processed to detect bright spots arising from individual molecules. Adherent molecules were defined by spots that can be linked in 4 or more consecutive frames and had an average diffusion constant below 10⁻⁸ cm²/sec over the duration of the trajectory.

### Results and Discussion

Multilayering. After priming the biotinylated lipid bilayer with a saturating concentration of avidin, actin filaments injected into solution gradually began forming an interspersed networked layer that leaves a significant fraction of the lipid bilayer exposed to the bathing solution. In effort to precisely control and monitor filament deposition and layer formation, filaments were injected in a series of sub steps (n) at a concentration below that required for layer saturation. Because the solution pH is below the pI of avidin, the exposed surface of the membrane maintains a positive electrostatic charge. This attracts the negative net charge of F-actin (at pH=7.5), which draws the polymer strands towards the interface. Upon contact, the biotin in the filaments combined with one of the four binding pockets in avidin and permanently adhered. Eventually, enough filaments deposited to form a fully saturated layer (Bₙ).

Prior to saturation, it was noted that a large majority of filaments associated with the surface and only a few remained in solution. That is, if the injected number of filaments is not sufficient to completely fill the available surface binding sites, all free filaments will settle and bind to the lipid surface over time. However, F-actin additions beyond n=2-5 showed diminished attraction for the linker-coated bilayer and the rate of filament surface binding slowed. When saturation occurred, a majority of the filaments permanently remained in solution above the surface, where they could be observed diffusing freely; they did not continue to settle on the surface over time.

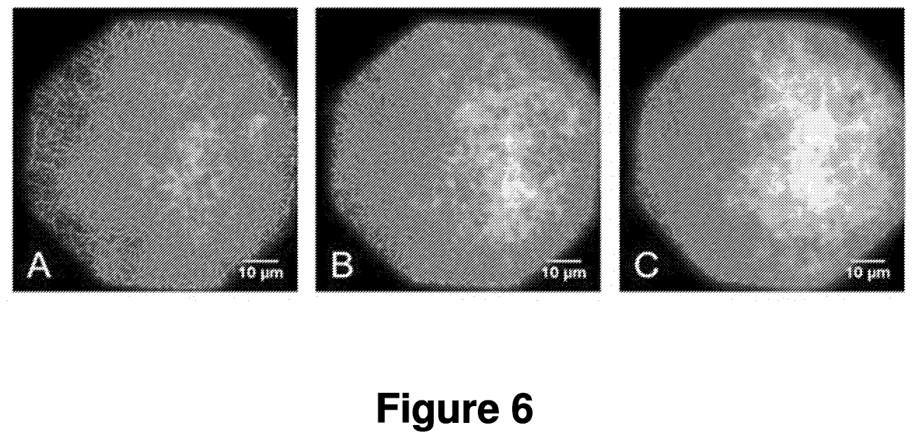

Following saturation, a chamber rinse (5× chamber volume) removed residual filaments and a second aliquot of linker (0.2 μg/ml) was added to coat the open biotinylation sites on the exposed F-actin filaments. This primed the top surface of the deposited filaments for cross-linking with additional F-actin. After washing away residual linker (10× chamber volume), actin filaments were again added to the chamber in small aliquots of 0.01 μM or 0.1 μM, which resulted in additional deposition. Eventually this second layer reached saturation once all accessible linker sites were occupied. This process was then repeated to form copolymer layers with a binding pattern (AB)_{N}. FIG. 6 shows the appearance of the resulting layers following full saturation of F-actin for N=1-3 multilayers that are linked to the lipid bilayer (FIG. 6B).

Layer Thickness. Individual actin filaments and actin bundles have been measured by atomic force microscopy to be 8 nm and 16 nm in diameter, respectively.⁴³ Biotin that is bound to streptavidin in opposing binding pockets can extend out to a distance of approximately 2.5 nm.⁴⁴ Therefore, it was estimated that the first saturated F-actin layer has a nominal height above the bilayer of 10.5-18.5 nm (assuming no bundling). Similarly, the second actin layer appeared to be of identical structure, which would increase the height of layers by another approximately 18.5 nm at the loci of filament overlap. Thus, theoretically, two F-actin layers form a porous network that occupies a 37-nm section above the lipid bilayer. Addition of a third layer extends the theoretical thickness at overlapping loci to over 55 nm.

Scanning-angle TIRF experiments of N=3 multilayer surfaces showed intensity trends that are indistinguishable from uncoated fluorescent lipid bilayers. Thus, it was concluded that layer deposition remained very thin, apparently below the axial resolution of the system. Presuming each cross-linked layer (N) occupies approximately 18.5 nm, a total of N=27 layers would be needed in order to achieve a thickness that approximates the evanescent wave penetration of the TIRF (approximately 500 nm). Based on prototype experiments, creating such a large number of layers appears possible. However, an automated microfluidic flow cell is desired but is not required to efficiently deliver materials and wash the surface so that thicker multilayer structures can be constructed.

Multilayer Structure Estimated Conduit Size. Inspection of images in FIG. 6 shows a few open areas on the N=1 saturated layer. These areas became less numerous as additional layers are added. Given the optical resolution of the microscope (approximately 500 nm), the largest openings in FIG. 6A were estimated to be ˜10° μm. Given the extensive filament overlap that was evident, many other passageways to the bilayer likely lie just below the resolution limit (e.g., 100-500 nm), making the estimated conduit size distribution approximately 10⁻¹-10⁰ μm for an N=1 structure. Because layers stack in 3D for a multilayered structure, the estimated conduit size distribution for an N=2 & 3 multilayered structure remains similar to that observed at the N=1 level, even though the filament density appeared to increase in FIGS. 6B and 6C.

Electrostatic Effects on Layer Architecture. Adjusting the surface electrostatics allowed control over both the deposition kinetics and the layer thickness. Avidin and neutravidin have isoelectric points of 10.5 and 6.3, respectively.^{45, 46} Reports of the pI of g-actin and F-actin range from 4.8-5.1.⁴⁷ Thus, at pH=7.5, both avidin and actin bear a significant charge; avidin's charge is positive and F-actin's charge is negative. These attractive forces impact both the rate of F-actin deposition and the structure of the layered filaments.

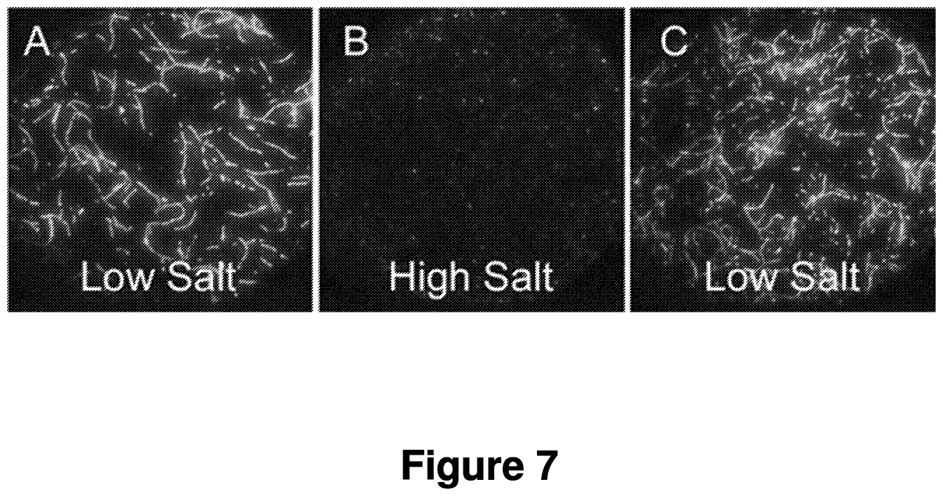

FIG. 7 shows a series of deposition measurements performed in pH=7.5 Tris-KCl buffer where the total ionic strength is adjusted with KCl. At low ionic strength (50 mM KCl), attractive charges are not screened effectively; thus, the negatively charged F-actin was drawn toward the positively charged avidin. Extended filaments, injected at a subsaturation concentration, were seen engaging in high affinity binding to the surface (FIG. 7A). After deposition stops, the filaments were photobleached to darkness, the chamber buffer was exchanged for a high salt concentration (1M KCl, pH=7.5), and another injection of F-actin was performed. At high salt (FIG. 7B), the attractive electrostatic forces appeared to be effectively screened, as only small fluorescent segments were added to the image over a long incubation period. After another chamber wash and a second round of photobleaching to darkness, a return to low salt conditions caused a third injection of F-actin to bind again (FIG. 7C). All these observations were attributed to electrostatic screening that either permits, or screens, long-range attractive forces to pull F-actin towards the surface, where high affinity linkages between biotin and actin are formed in the remaining uncovered spaces of the avidin-coated lipid bilayer.

Multilayer Structures on Microcavities in MECA4-Fluo Bilayer Array. Lipid bilayers are formed over the MECA4-Fluo microcavities following standard procedures. The Orbit Mini provides tools for measuring and tracking bilayer capacitance over time. This feature was employed as a diagnostic guide for identifying properly formed bilayers and multilayer support structures. Typical capacitance readings for properly formed planar lipid bilayers (without a multilayered support) range from 50-100 pF. Given the size of the microcavity apertures (150 μm dia.), this corresponds to specific capacitances in the range of ˜0.3-0.8 μF/cm², which favorably matches values found in the literature for functional bilayers (0.4-1 μF/cm²).^{36, 37} These capacitances are achieved in >75% of bilayer formation attempts and are also readily permeable by αHL. Generally, microcavities exhibiting a capacitance of less than approximately 25 pF were ignored for imaging experiments. Small capacitances for lipid bilayers are most likely due to an excessively large annulus or residual lipid material retained within the microcavity. After construction of a multilayered support on top of the bilayer (e.g., N=3), capacitance values drop by 5-50%, which indicates a properly formed multilayer-coated lipid bilayer.

During imaging experiments on individual microcavities, membrane electrical measurements continued simultaneously. Typically, the applied voltage is changed to a DC potential to monitor transmembrane conductivity during imaging.

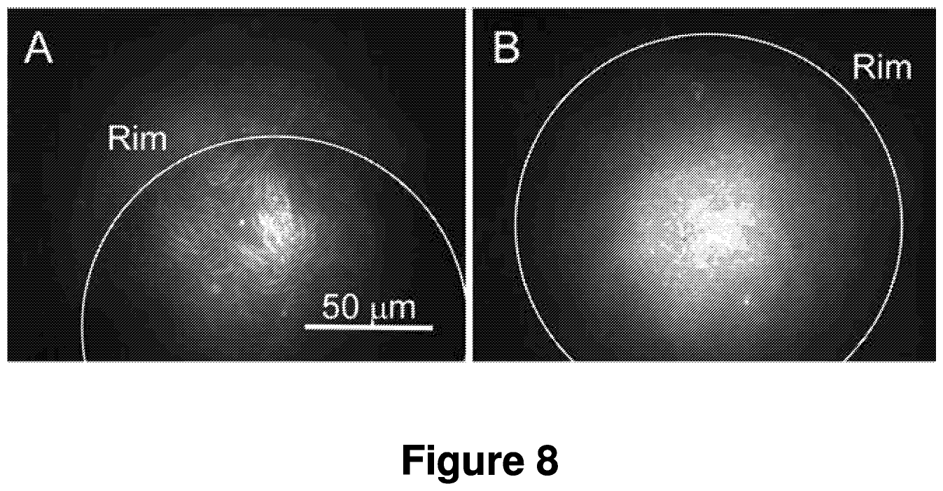

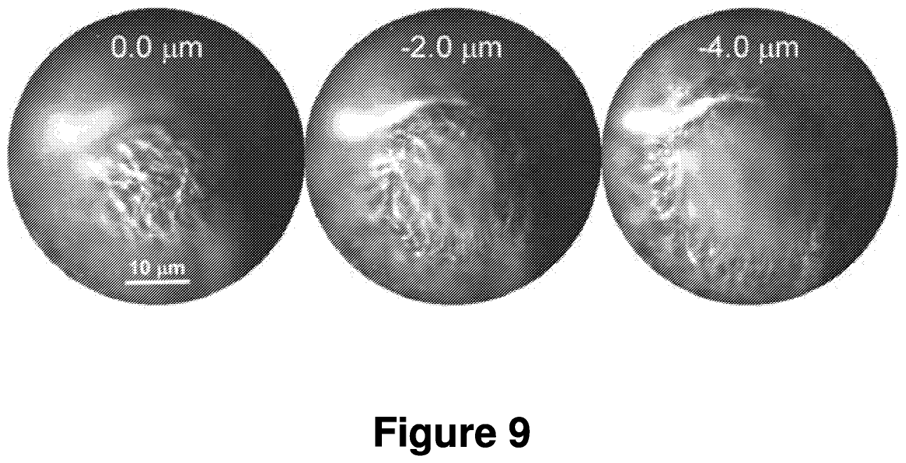

As can be seen in FIG. 8, stationary actin filaments were readily visible on top of the microcavity. Background scatter and fluorescence from the chip substrate enabled the aperture rim to be brought into focus as demarked in the images. FIG. 8A shows the appearance of a saturated layer at N=1, prior to the addition of linker that began the construction of layer 2 in the multilayer structure. Single layers of filaments appeared slightly less dense than the equivalent layers observed at N=1 on glass supported bilayers (FIG. 6A). The addition of two more layers increased the filament surface density significantly (FIG. 8B), creating a uniform coating over the lipid bilayer, as well as portions of the chip substrate that contain the microcavities. In a majority of cases, the coated bilayers remained flat to within the axial resolution of the microscope. Such is the case in FIG. 8. However, occasionally structures were observed with concave and convex curvatures that permitted only a portion of the membrane structure to be brought into focus. This curvature can be visualized by scanning in the z direction. FIG. 9 demonstrates a multilayer structure formed with a convex curvature, where the apex of the membrane is positioned above the microcavity rim by approximately 10 μm.

Additionally, an axial scan performed along the entire depth of the microcavity revealed a series of images that is consistent with an aqueous-filled cavity covered by a thin multilayer coated bilayer. The axial resolution of the optics is approximately 2 μm. FIG. 10 shows a series of images starting at the bottom of the microcavity, where small granules of the Ag/AgCl microelectrode come into focus. This surface fluoresced brightly with temporary flashes that presumably arise from surface enhanced Raman and/or fluorescence effects. Moving up from the bottom of the cavity by approximately 7 μm, only out-of-focus background fluorescence from the microelectrode and faint features from fluorescent actin on the bilayer at the top of the microcavity appear. At 13 μm above the bottom surface, filaments from a flat multilayer coated bilayer came into focus. A semicircular portion of the actin filaments had been previously photobleached to help mark the axial location of the multilayer structure, while the distinct boundary between bleached and non-bleached regions indicates the filaments remain relatively stationary in the intertwined network. Moving above the membrane network by another 6 μm showed no spatial contrast and only dim out-of-focus fluorescence that is typical of bulk aqueous solution.

Multilayer Permeability and Ion-Selective Transport. The relative 2D openness of the filamentous actin web creates large water-filled conduits within the layered structure. This porosity and the generally thin dimensions of a multilayer structure should permit high molecular permeability. Thus, molecules in the surrounding aqueous environment can diffuse to the lipid bilayer surface with little inhibition. Such permeability is critical for many applications, especially when ion channels or nanopores need to be inserted into the bilayer. Both αHL and BSA molecules were used to test multilayer structure permeability.

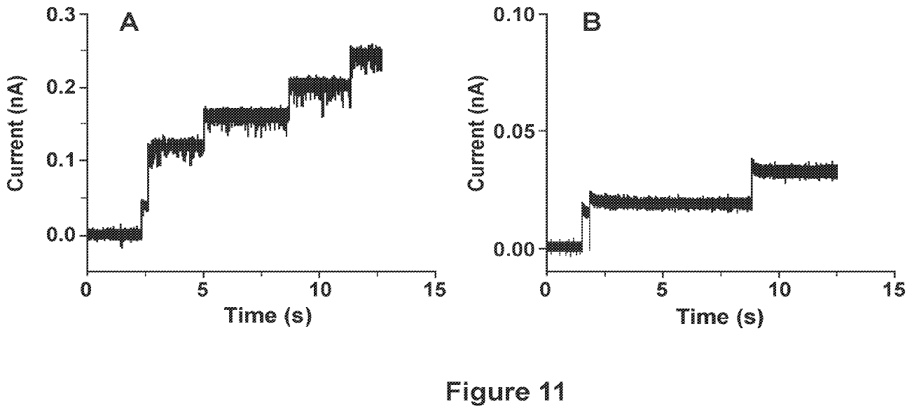

To test permeability, multilayer structures (N=1 or 3) were formed using the (A-wash-Bₙ-wash)_{N} procedure with an avidin linker, 0.1-1.0 mol % biotinylated lipid in the bilayer, and pH=7.5 Tris-KCl buffer (1M KCl). Multilayer-coated (N=3) bilayers formed on the MECA4-Fluo chip were tested with αHL using a 50 mV DC potential. FIG. 11 shows typical results from one microcavity in the array that does not possess a multilayer coating. Within seconds of αHL addition, nanopore insertion in the bilayer was observed as discrete jumps in conductivity. Similar insertion time lags (i.e., seconds) were observed for both N=1 and N=3 multilayers. Thus, it was concluded that an N=3 multilayer constructed with avidin is rapidly permeable. Essentially, lag times in the multilayered structure cannot be distinguished from an uncoated lipid bilayer.

FIG. 11 shows insertion currents from an (N=3) membrane, as well as nanopore insertions from an uncoated lipid bilayer. Nanopore insertion currents for the uncoated membranes produce and average of approximately 50 pA/insertion (100 insertions). However, nanopores from an N=3 multilayer structure (20 insertions) exhibited a significantly lower conductance. The nature of this difference is not fully understood, but given the significant electrostatic effects observed during the creation of the multilayer, it is likely that charge held within the layered structure altered the effective transmembrane potential that drives ions through the nanopore, or charge screening impacted the flux of ions that were available for transport through the nanopore. This observation is likely related to ion-selective permeability of the multilayer structure. Indeed, synthetic polyelectrolyte multilayer thin films with layered electrostatic structure similar to that of the multilayers formed herein have been shown to possess ion-selective transport. 48 In any case, the comparable lag times (seconds) for insertion after injection for all membrane types (uncoated and multilayered) indicated that the multilayer structure does little to prohibit diffusion to the bilayer, as well as nanopore self-assembly in the bilayer. Thus, the data herein demonstrate that a multilayer support of up to N=3 is highly permeable, and it is expected that multilayer supports comprising additional layers (e.g., N=5, 10, or 100) are also highly permeable.

Multilayer Permeability Rate Probed by Single Molecule Diffusion. Bovine serum albumin is a large 66 kDa protein with dimensions of a prolate ellipsoid (14 nm×4 nm×4 nm) and an isoelectric point of 4.7. It is known to undergo nonspecific association with phosphocholine lipid headgroups. BSA is also significantly larger than αHL monomers. So, it provided a more challenging, high-molecular weight permeability test for the multilayer. If BSA penetrates the multilayer structure and binds to lipid, it should move laterally within the plane of the bilayer, along with the lipid molecules to which it is bound. Detection of continuous lateral movement signals full penetration through the multilayer structure and contact with the lipid bilayer.

Using the evanescent field from TIRF illumination, the number of lateral single-molecule diffusion trajectories was monitored before and after injection of BSA into solution above the multilayer. Comparing the number of molecules before and after injection acted as a secondary means to assess the permeability of the multilayer structure, both in terms of particle size and rate of permeation.

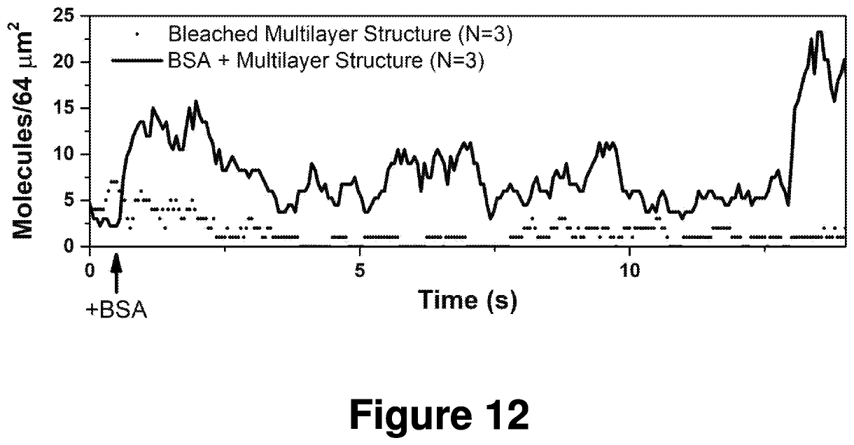

FIG. 12 recorded the number of single-molecule diffusion trajectories present on the bilayer before and after BSA was injected into the sample chamber at a concentration of 1 nM. First, the multilayer structure was bleached by prolonged exposure to the laser. Then a background recording was performed over a 64 prn 2 region. As can be seen, the number of single molecules diffusing in the bilayer plane increased sharply over the background within approximately 75 ms of BSA injection. This indicated that multilayer structure permeation occurred with little inhibition. In fact, the time to appearance of BSA molecules on the N=3 multilayer-coated surface is indistinguishable from the time to appearance of BSA on bilayers without any multilayer structures.

### CONCLUSION

Described herein is a chip-based lipid bilayer array that possesses a stratified coating of multiple cross-linked layers. The methodology used to form the multilayered structure can be employed for nanopore sensing or ion channel applications, or also in expanded forms with much larger arrays. The closed structure of chip's microcavities adds significant durability by effectively eliminating the solution pressure gradient across the bilayer. Multiple cross-linked layers of F-actin that are chemically linked to the bilayer formed on top of the microcavity, further enhances stability and durability in a manner similar to the cytoskeleton of living cells.

Actin deposition rates can be altered by controlling the electrostatic interactions between filaments and linkers in order to increase or decrease the local electric field strength between layers. The time chosen for deposition (approximately 10 minutes per adlayer) was arbitrary. Although multilayered structures of up to N=3 were tested, multilayer structures of arbitrary thickness (N of about 10,000 or more) can be formed and are contemplated herein.

Finally, conduit sizes are adjustable by altering the biotinylation density of both the lipid bilayer and the F-actin. The biotinylation densities employed herein (0.1-1 mole % in the bilayer and up to 20 mol % in F-actin) appeared to produce conduit sizes in the range of 10⁻¹-10⁰ μm. Furthermore, the network structure did not prohibit molecular access to the bilayer, as evidenced by αHL insertion. Experiments with other linkers from the avidin family that possess significantly different isoelectric points and enable attractive and repulsive electrostatic interactions are ongoing.

### REFERENCES

(1) Cao, C.; Long, Y.-T. Biological Nanopores: Confined Spaces for Electrochemical Single-Molecule Analysis. Acc. Chem. Res. 2018, 51 (2), 331-341. https://doi.org/10.1021/a cs.accounts.7b00143.
(2) Cressiot, B.; Ouldali, H.; Pastoriza-Gallego, M.; Bacri, L.; Van der Goot, F. G.; Pelta, J. Aerolysin, a Powerful Protein Sensor for Fundamental Studies and Development of Upcoming Applications. ACS Sens. 2019, 4 (3), 530-548. https://doi.org/10.1021/a cssensors.8b01636.
(3) Kasianowicz, J. J.; Balijepalli, A. K.; Ettedgui, J.; Forstater, J. H.; Wang, H.; Zhang, H.; Robertson, J. W. Analytical Applications for Pore-Forming Proteins. Biochim. Biophys. Acta 2016, 1858 (3), 593-606.
(4) Misawa, N., Toshihisarlakeuchi, Shoji. Membrane Protein-Based Biosensors. Interface J. R. Soc. Interface 2018, 15 (141), 20170952.
(5) Liu, L.; Wu, H. C. DNA-Based Nanopore Sensing. Angew. Chem. 2016, 55 (49), 15216-15222.
(6) Schmidt, J. Membrane Platforms for Biological Nanopore Sensing and Sequencing. Curr. Opin. Inbiotechnology 2016, 39, 17-27.
(7) Yan, H.; Zhou, D.; Shi, B.; Zhang, Z.; Tian, H.; Yu, L.; Wang, Y.; Guan, X.; Wang, Z.; Wang, D. Slowing down DNA Translocation Velocity Using a LiCl Salt Gradient and Nanofiber Mesh. Eur. Biophys. J. 2019, 48 (3), 261-266. https://doi.org/10.1007/s00249-019-01350-x.
(8) Asandei, A.; Mereuta, L.; Park, J.; Seo, C. H.; Park, Y.; Luchian, T. Nonfunctionalized PNAs as Beacons for Nucleic Acid Detection in a Nanopore System. ACS Sens. 2019, 4 (6), 1502-1507. https://doi.org/10.1021/a cssensors.9b00553.
(9) Chen, K.; Kong, J.; Zhu, J.; Ermann, N.; Predki, P.; Keyser, U. F. Digital Data Storage Using DNA Nanostructures and Solid-State Nanopores. Nano Lett. 2019, 19 (2), 1210-1215. https://doi.org/10.1021/a cs.nanolett.8b04715.
(10) Ivica, J.; Williamson, P. T. F.; de Planque, M. R. R. Salt Gradient Modulation of MicroRNA Translocation through a Biological Nanopore. Anal. Chem. 2017, 89 (17), 8822-8829. https://doi.org/10.1021/a cs.analchem.7b01246.
(11) Yang, J.; Wang, Y.-Q.; Li, M.-Y.; Ying, Y.-L.; Long, Y.-T. Direct Sensing of Single Native RNA with a Single-Biomolecule Interface of Aerolysin Nanopore. Langmuir 2018, 34 (49), 14940-14945. https://doi.org/10.1021/a cs.langmuir.8b03264.
(12) Asandei, A.; Dragomir, I.; Di Muccio, G.; Chinappi, M.; Park, Y.; Luchian, T. Single-Molecule Dynamics and Discrimination between Hydrophilic and Hydrophobic Amino Acids in Peptides, through Controllable, Stepwise Translocation across Nanopores. Polymers 2018, 10 (8), 885. https://doi.org/10.3390/polym10080885.
(13) Bonome, E., FabiolChinappi, Mauro. Translocation Intermediates of Ubiquitin through an α-Hemolysin Nanopore: Implications for Detection of Post-Translational Modifications. Nanoscale 2019, 11 (20), 9920-9930.
(14) Fahie, M. A.; Yang, B.; Mullis, M.; Holden, M. A.; Chen, M. Selective Detection of Protein Homologues in Serum Using an OmpG Nanopore. Anal. Chem. 2015, 87 (21), 11143-11149.
(15) Hoogerheide, D. P.; Gurney, P. A.; Rostovtseva, T. K.; Bezrukov, S. M. Real-Time Nanopore-Based Recognition of Protein Translocation Success. Biophys. J. 2018, 114 (4), 772-776. https://doi.org/10.1016/j.bpj.2017.12.019.
(16) Robertson, J. W. F.; Reiner, J. E. The Utility of Nanopore Technology for Protein and Peptide Sensing. PROTEOMICS 2018, 18 (18), 1800026. https://doi.org/10.1002/pmic.201800026.
(17) Houghtaling, J.; List, J.; Mayer, M. Nanopore-Based, Rapid Characterization of Individual Amyloid Particles in Solution: Concepts, Challenges, and Prospects. Small 2018, 14 (46), 1802412. https://doi.org/10.1002/sm11.201802412.
(18) Liu, L.; Li, T.; Zhang, S.; Song, P.; Guo, B.; Zhao, Y.; Wu, H.-C. Simultaneous Quantification of Multiple Cancer Biomarkers in Blood Samples through DNA-Assisted Nanopore Sensing. Angew. Chem. Int. Ed. 2018, 57 (37), 11882-11887. https://doi.org/10.1002/a nie.201803324.
(19) Zhang, Z.; Li, T.; Sheng, Y.; Liu, L.; Wu, H.-C. Enhanced Sensitivity in Nanopore Sensing of Cancer Biomarkers in Human Blood via Click Chemistry. Small 2019, 15 (2), 1804078. https://doi.org/10.1002/sm11.201804078.
(20) Zhao, Y.; Liu, L.; Tu, Y.; Wu, H. Investigating the Effect of Mono- and Multivalent Counterions on the Conformation of Poly(Styrenesulfonic Acid) by Nanopores. ELECTROPHORESIS 2019, 40 (16-17), 2180-2185. https://doi.org/10.1002/elps.201800539.
(21) Cai, S.; Sze, J. Y. Y.; Ivanov, A. P.; Edel, J. B. Small Molecule Electro-Optical Binding Assay Using Nanopores. Nat. Commun. 2019, 10 (1), 1797. https://doi.org/10.1038/s41467-019-09476-4.
(22) Liu, L.; Fang, Z.; Zheng, X.; Xi, D. Nanopore-Based Strategy for Sensing of Copper(II) Ion and Real-Time Monitoring of a Click Reaction. ACS Sens. 2019, 4 (5), 1323-1328. https://doi.org/10.1021/a cssensors.9b00236.
(23) Wang, L.; Yao, F.; Kang, X. Nanopore Single-Molecule Analysis of Metal lon—Chelator Chemical Reaction. Anal. Chem. 2017, 89 (15), 7958-7965. https://doi.org/10.1021/a cs.analchem.7b01119.
(24) Hsu, W.-L.; Hwang, J.; Daiguji, H. Theory of Transport-Induced-Charge Electroosmotic Pumping toward Alternating Current Resistive Pulse Sensing. ACS Sens. 2018, 3 (11), 2320-2326. https://doi.org/10.1021/a cssensors.8b00635.
(25) Xia, X.; Jiang, X.; Chen, Y. Nanoanalysis and Nanomaterials-based Separation and Devices. ELECTROPHORESIS 2019, 40 (16-17), 2010-2010. https://doi.org/10.1002/elps.201970115.
(26) Zhang, J.; Li, Z.; Zhan, K.; Sun, R.; Sheng, Z.; Wang, M.; Wang, S.; Hou, X. Two Dimensional Nanomaterial-based Separation Membranes. ELECTROPHORESIS 2019, 40 (16-17), 2029-2040. https://doi.org/10.1002/elps.201800529.
(27) Liu, L.; Xie, J.; Li, T.; Wu, H.-C. Fabrication of Nanopores with Ultrashort Single-Walled Carbon Nanotubes Inserted in a Lipid Bilayer. Nat. Protoc. 2015, 10 (11), 1670-1678. https://doi.org/10.1038/nprot.2015.112.
(28) Wang, Y.-Q.; Cao, C.; Ying, Y.-L.; Li, S.; Wang, M.-B.; Huang, J.; Long, Y.-T. Rationally Designed Sensing Selectivity and Sensitivity of an Aerolysin Nanopore via Site-Directed Mutagenesis. ACS Sens. 2018, 3 (4), 779-783. https://doi.org/10.1021/a cssensors.8b00021.
(29) Panahi, Y.; Farshbaf, M.; Mohammadhosseini, M.; Mirahadi, M.; Khalilov, R.; Saghfi, S.; Akbarzadeh, A. Recent Advances on Liposomal Nanoparticles: Synthesis, Characterization and Biomedical Applications. Artif. Cells Nanomedicine Biotechnol. 2017, 45 (4), 788-799. https://doi.org/10.1080/21691401.2017.1282496.
(30) Obergrussberger, A. F., Sonja|Becker, Nadine|Brüggemann, Andrea|Fertig, Niels|Möller, Clemens. Novel Screening Techniques for Ion Channel Targeting Drugs. Channels 2015, 9 (6), 367-375.
(31) Yao, F.; Duan, J.; Wang, Y.; Zhang, Y.; Guo, Y.; Guo, H.; Kang, X. Nanopore Single-Molecule Analysis of DNA—Doxorubicin Interactions. Anal. Chem. 2015, 87 (1), 338-342. https://doi.org/10.1021/a c503926g.
(32) Beddow, J. A.; Peterson, I. R.; Heptinstall, J.; Walton, D. J. Reconstitution of Nicotinic Acetylcholine Receptors into Gel-Protected Lipid Membranes. Anal. Chem. 2004, 76 (8), 2261-2265. https://doi.org/10.1021/a c0350514.
(33) Jeon, T. J.; Malmstadt, N.; Schmidt, J. J. Hydrogel-Encapsulated Lipid Membranes. J. Am. Chem. Soc. 2006, 128 (1), 42-43.
(34) Jeon, T.-J., NoahlPoulos, JasonlSchmidt, Jacob. Black Lipid Membranes Stabilized through Substrate Conjugation to a Hydrogel. Biointerphases 2008, 3 (2), FA96-FA100.
(35) Bright, L. K.; Baker, C. A.; Branstrom, R.; Saavedra, S. S.; Aspinwall, C. A. Methacrylate Polymer Scaffolding Enhances the Stability of Suspended Lipid Bilayers for Ion Channel Recordings and Biosensor Development. ACS Biomater. Sci. Eng. 2015, 1 (10), 955-963.
(36) Burden, D. L.; Kim, D.; Cheng, W.; Chandler Lawler, E.; Dreyer, D. R.; Keranen Burden, L. M. Mechanically Enhancing Planar Lipid Bilayers with a Minimal Actin Cortex. Langmuir 2018, 34 (37), 10847-10855. https://doi.org/10.1021/a cs.langmuir.8b01847.
(37) Andersson, J.; Koper, I. Tethered and Polymer Supported Bilayer Lipid Membranes: Structure and Function. Membranes 2016, 6 (2).
(38) Sugihara, K.; Voros, J.; Zambelli, T. A Gigaseal Obtained with a Self-Assembled Long-Lifetime Lipid Bilayer on a Single Polyelectrolyte Multilayer-Filled Nanopore. ACS Nano 2010, 4 (9), 5047-5054. https://doi.org/10.1021/nn100773q.
(39) Watkins, E. B.; El-khouri, R. J.; Miller, C. E.; Seaby, B. G.; Majewski, J.; Marques, C. M.; Kuhl, T. L. Structure and Thermodynamics of Lipid Bilayers on Polyethylene Glycol Cushions: Fact and Fiction of PEG Cushioned Membranes. Langmuir ACS J. Surf. Colloids 2011, 27 (22), 13618-13628.
(40) Shenoy, D. K.; Barger, W. R.; Singh, A.; Panchal, R. G.; Misakian, M.; Stanford, V. M.; Kasianowicz, J. J. Functional Reconstitution of Protein Ion Channels into Planar Polymerizable Phospholipid Membranes. Nano Lett. 2005, 5 (6), 1181-1185.
(41) Vogel, S. Minimal Systems to Study Membrane—Cytoskeleton Interactions. Curr. Opin. Biotechnol. 2012, 23 (5), 758-765.
(42) Vogel. The Design of MACs (Minimal Actin Cortices). Cytoskeleton 2013, 70 (11), 706-717.
(43) Nöding, H.; Schein, M.; Reinermann, C.; Dörrer, N.; Kürschner, A.; Geil, B.; Mey, I.; Heussinger, C.; Janshoff, A.; Steinem, C. Rheology of Membrane-Attached Minimal Actin Cortices. J. Phys. Chem. B 2018,122 (16), 4537-4545.
(44) Le Trong, I.; Wang, Z.; Hyre, D. E.; Lybrand, T. P.; Stayton, P. S.; Stenkamp, R. E. Streptavidin and Its Biotin Complex at Atomic Resolution. Acta Crystallogr Sect D 67,813-821.
(45) Barinaga-Rementeria Ramirez, I.; Ekblad, L.; Jergil, B. Affinity Partitioning of Biotinylated Mixed Liposomes: Effect of Charge on Biotin—NeutrAvidin Interaction. J. Chromatogr. B. Biomed. Sci. App. 2000,743 (1-2), 389-396. https://doi.org/10.1016/50378-4347(00)00065-7.
(46) Zocchi, A.; Marya Jobé, A.; Neuhaus, J.-M.; Ward, T. R. Expression and Purification of a Recombinant Avidin with a Lowered Isoelectric Point in Pichia Pastoris. Protein Expr. Purif. 2003, 32 (2), 167-174. https://doi.org/10.1016/j.pep.2003.09.001.
(47) DEBAIN|Debain, V. Les Points Isoélectriques de La F-Actine et de La F-Actine Dépolymérisée. Biochim. Biophys. Acta 1956, 21 (1), 182-183.
(48) Stanton, B. W.; Harris, J. J.; Miller, M. D.; Bruening, M. L. Ultrathin, Multilayered Polyelectrolyte Films as Nanofiltration Membranes. Langmuir 2003, 19 (17), 7038-7042. https://doi.org/10.1021/Ia034603a.

All documents cited herein are, in relevant part, incorporated herein by reference.

What is claimed is:

1. A device comprising a lipid bilayer that is linked to at least two layers of interconnected polymer filaments, wherein the lipid bilayer is attached to a substrate.

2. The device of claim 1, wherein each of the at least two layers of interconnected polymer filaments is from about 8 to about 16 nanometers (nm) in thickness.

3. The device of claim 1 or claim 2, wherein each of the at least two layers of interconnected polymer filaments bears a net positive electrostatic charge, a net negative electrostatic charge, or no electrostatic charge.

4. The device of any one of claims 1-3, wherein each of the at least two layers of interconnected polymer filaments withstands at least about 55 Pascals (Pa) of pressure without significant deformation.

5. The device of any one of claims 1-4, wherein the electrical resistivity of the device is from about 2 to about 100 gigaohms (Gohm), or from about 10 to about 100 Gohm.

6. The device of any one of claims 1-5, wherein the at least two layers of interconnected polymer filaments are chemically linked to each other.

7. The device of any one of claims 1-6, wherein the chemical link is a covalent link, a non-covalent link, or an ionic link.

8. The device of any one of claims 1-7, wherein each of the at least two layers of interconnected polymer filaments comprises a cross-linking site.

9. The device of claim 8, wherein from about 0.001% to about 100% of the surface of each of the at least two layers of interconnected polymer filaments comprises an anchor.

10. The device of any one of claims 1-9, wherein each of the at least two layers of interconnected polymer filaments comprises a polypeptide, an oligonucleotide, an oligosaccharide, a polymer gel, hydrogel, or a combination thereof.

11. The device of claim 10, wherein each of the at least two layers of interconnected polymer filaments comprises a polypeptide.

12. The device of claim 11, wherein the polypeptide is a cytoskeletal polypeptide.

13. The device of claim 12, wherein the cytoskeletal polypeptide is a catenin, an intermediate filament protein, a microfilament protein, or a microtubule protein.

14. The device of claim 13, wherein the catenin is alpha catenin, beta catenin, or gamma catenin.

15. The device of claim 13, wherein the intermediate filament protein is desmin, glial fibrillary acidic protein, keratin, nestin, or vimentin.

16. The device of claim 13, wherein the microfilament protein is actin, actinin, filamin, gelsolin, myosin, profilin, tensin, tropomyosin, troponin, or a derivative thereof.

17. The device of claim 13, wherein the microtubule protein is dynein, tubulin, or kinesin.

18. The device of any one of claims 1-17, wherein the at least two layers of interconnected polymer filaments are linked to each other through a cross-linking site.

19. The device of claim 18, wherein the cross-linking site comprises (i) biotin and streptavidin; (ii) spectrin; (iii) avidin, neutravidin, or a biotin binding protein; (iv) a bridge protein from the ERM family; (v) a bridge protein from the formin family; (vi) a transmembrane glycoprotein; or (vii) digoxygenin and an antibody directed against digoxygenin.

20. The device of any one of claims 1-19, wherein the at least two layers of interconnected polymer filaments are chemically linked to the lipid bilayer.

21. The device of claim 20, wherein the chemical link is a covalent link, a non-covalent link, or an ionic link.

22. The device of any one of claims 1-21, wherein the lipid bilayer comprises an anchor.

23. The device of claim 22, wherein from about 0.001% to about 100% of the lipid bilayer surface comprises an anchor.

24. The device of any one of claims 1-23, wherein at least one of the at least two layers of interconnected polymer filaments are linked to the lipid bilayer through a cross-linking site.

25. The device of claim 24, wherein the cross-linking site comprises (i) biotin and streptavidin; (ii) spectrin; (iii) avidin, neutravidin, or a biotin binding protein; (iv) a bridge protein from the ERM family; (v) a bridge protein from the formin family; (vi) a transmembrane glycoprotein; or (vii) digoxygenin and an antibody directed against digoxygenin.

26. The device of any one of claims 1-25, wherein the substrate comprises an aperture.

27. The device of claim 26, wherein the aperture is from about 10 nanometers (nm) to about 1000 microns (μm) in diameter.

28. The device of any one of claims 1-27, wherein the substrate is a polymer resin, glass, or a semiconductor.

29. The device of any one of claims 1-28, wherein the lipid bilayer spans the aperture.

30. The device of any one of claims 26-29, wherein the aperture is about 50 microns to about 500 microns in diameter, or from about 100 nm to about 1 millimeter.

31. The device of any one of claims 1-30, further comprising at least one ion channel forming at least one pore through the lipid bilayer.

32. The device of any one of claims 1-31, wherein each of the at least two layers comprises a conduit between the interconnected polymer filaments.

33. The device of claim 32, wherein the conduit is from about 10⁻³ to about 10⁰ microns (μm) in diameter.

34. The device of any one of claims 29-33, wherein the device comprises a plurality of apertures.

35. The device of any one of claims 31-34, wherein the device comprises one pore per aperture.

36. The device of claim 35, wherein the device comprises about 10, or about 20, or about 30, or about 40, or about 50, or about 60, or about 70, or about 80, or about 90, or about 100, or about 200, or about 500, or about 1000, or about 2000, or about 3000, or about 5000, or about 7000, or about 10000 apertures.

37. The device of any one of claims 31-36, wherein the ion channel is a protein ion channel, Staphylococcus aureus alpha-hemolysin, Bacillus anthracis protective antigen 63, gramicidin, MspA (Mycobacterium smegmatis), OmpF porin, Kapton, OmpG, ClyA (Salmonella typhimurium), a non-naturally occurring compound, or derivatives thereof.

38. The device of any one of claims 31-37, further comprising a molecular motor, wherein said motor is adjacent to the at least one pore and is capable of moving a polymer with respect to the at least one pore.

39. The device of claim 38, wherein the molecular motor comprises a DNA polymerase, a RNA polymerase, a ribosome, an exonuclease, or a helicase and said polymer is a polynucleotide.

40. The device of claim 39, wherein the DNA polymerase is selected from E. coli DNA polymerase I, E. coli DNA polymerase I Large Fragment (Klenow fragment), phage T7 DNA polymerase, Phi-29 DNA polymerase, Thermus aquaticus (Taq) DNA polymerase, Thermus flavus (Tfl) DNA polymerase, Thermus Thermophilus (Tth) DNA polymerase, Thermococcus litoralis (Tli) DNA polymerase, Pyrococcus furiosus (Pfu) DNA polymerase, Bacillus stearothermophilus (Bst) DNA polymerase, AMV reverse transcriptase, MMLV reverse transcriptase, and HIV-1 reverse transcriptase.

41. The device of claim 39, wherein the RNA polymerase is selected from T7 RNA polymerase, T3 RNA polymerase, SP6 RNA polymerase, and E. coli RNA polymerase.

42. The device of claim 39, wherein the exonuclease is selected from exonuclease Lambda, T7 Exonuclease, Exo III, RecJ1 Exonuclease, Exo I, and Exo T.

43. The device of claim 39, wherein the helicase is selected from E-coli bacteriophage T7 gp4 and T4 gp41 gene proteins, E. coli protein DnaB, E. coli protein RuvB, and E. coli protein rho.

44. The device of any one of claims 1-43, wherein the lipid bilayer comprises a plurality of lipid groups comprising one or more of diphytanoyl 1,2,-diacyl-sn-glycero-3-[phosphor-L-serine] (DiPHyPC), 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE).

45. The device of any one of claims 1-44, wherein each of the at least two layers of interconnected polymer filaments has a density that is from about 0.01 filaments per μm 2 to about 10,000 filaments per μm².

46. The device of claim 45, wherein density of the at least two layers of interconnected polymer filaments is about the same.

47. The device of claim 45, wherein the at least two layers of interconnected polymer filaments have different densities.

48. The device of any one of claims 1-47, wherein the device comprises three layers of interconnected polymer filaments.

49. The device of any one of claims 1-47, wherein the device comprises from about 2 to about 10,000 or more layers of interconnected polymer filaments.

50. The device of any one of claims 1-49, wherein the device is permeable to a molecule having a size radius of between about 50 picometers (pm) to about 500 nanometers (nm).

51. The device of any one of claims 1-50, wherein the device is permeable to a molecule having a molecular weight from about 10 to about 1,000,000 daltons.

52. The device of any one of claims 1-51, wherein the device is permeable to a molecule having a charge of from about −2×10⁶ to about +2×10⁶, or from about −50 to about +50.

53. A method of analyzing a target polymer comprising contacting the target polymer with the device of any one of claims 31-52 to allow the target polymer to move with respect to the at least one pore to produce a signal, and monitoring the signal corresponding to the movement of the target polymer with respect to the pore, thereby analyzing the target polymer.

54. The method of claim 53, wherein the signal monitoring comprises measuring a monomer-dependent characteristic of the target polymer while the target polymer moves with respect to the pore.

55. The method of claim 54, wherein the monomer dependent property is the identity of a monomer or the number of monomers in the polymer.

56. The method of any one of claims 53-55, further comprising altering the rate of movement of the polymer before, during, or after the signal monitoring.

57. The method of any one of claims 53-56, wherein the target polymer is an oligonucleotide, a polypeptide, or an oligosaccharide.

58. The method of claim 57, wherein the oligonucleotide is DNA.

59. The method of any one of claims 53-58, wherein the analyzing comprises a chemical characterization.

60. The method of claim 59, wherein the chemical characterization is a characterization of DNA, a synthetic polymer, a small molecule, or an ion.

61. The method of claim 60, wherein the characterization of DNA comprises nucleotide sequencing or genotyping.

62. A method of forming a device comprising:
(a) providing a lipid bilayer, the lipid bilayer comprising a first anchor, wherein the lipid bilayer is associated with a substrate, the substrate comprising an aperture and an electrode;
(b) applying a linker molecule;
(c) providing a first layer of polymer filaments comprising a second anchor, thereby creating a cross-linking site between the first anchor, the linker molecule, and the second anchor, thereby linking the lipid bilayer to the first layer of polymer filaments;
(d) applying the linker molecule;
(e) providing a second layer of polymer filaments comprising a third anchor, thereby creating a cross-linking site between the second anchor, the linker molecule, and the third anchor, thereby linking the first layer of polymer filaments to the second layer of polymer filaments; and
(f) inserting a pore into the lipid bilayer, thereby forming the device.

63. The method of claim 62, further comprising applying the linker molecule between steps (e) and (f); and
providing a third layer of polymer filaments comprising a fourth anchor, thereby creating a cross-linking site between the third anchor, the linker molecule, and the fourth anchor, thereby linking the second layer of polymer filaments to the third layer of polymer filaments.

64. The method of claim 62 or claim 63, wherein each of the first layer of polymer filaments and the second layer of polymer filaments is from about 8 to about 16 nanometers (nm) in thickness.

65. The method of any one of claims 62-64, wherein the third layer of polymer filaments is from about 8 to about 16 nanometers (nm) in thickness.

66. The method of any one of claims 62-65, wherein each of the first layer of polymer filaments and the second layer of polymer filaments withstands at least about 55 Pascals (Pa) of pressure without significant deformation.

67. The method of any one of claims 62-66, wherein the third layer of polymer filaments withstands at least about 55 Pascals (Pa) of pressure without significant deformation.

68. The method of any one of claims 62-67, wherein the electrical resistivity of the device is from about 10 to about 100 gigaohms (Gohm).

69. The method of any one of claims 62-68, wherein from about 0.001% to about 100% of the surface of each of the first layer of polymer filaments, the second layer of polymer filaments, and the third layer of polymer filaments each comprises an anchor.

70. The method of any one of claims 62-69, wherein the first layer of polymer filaments, the second layer of polymer filaments, and the third layer of polymer filaments each comprises a polypeptide, an oligonucleotide, an oligosaccharide, a polymer gel, hydrogel, or a combination thereof.

71. The method of claim 70, wherein the first layer of polymer filaments, the second layer of polymer filaments, and/or the third layer of polymer filaments each comprises a polypeptide.

72. The method of claim 71, wherein the polypeptide is a cytoskeletal polypeptide.

73. The method of claim 72, wherein the cytoskeletal polypeptide is a catenin, an intermediate filament protein, a microfilament protein, or a microtubule protein.

74. The method of claim 73, wherein the catenin is alpha catenin, beta catenin, or gamma catenin.

75. The method of claim 73, wherein the intermediate filament protein is desmin, glial fibrillary acidic protein, keratin, nestin, or vimentin.

76. The method of claim 73, wherein the microfilament protein is actin, actinin, filamin, gelsolin, myosin, profilin, tensin, tropomyosin, troponin, or a derivative thereof.

77. The method of claim 73, wherein the microtubule protein is dynein, tubulin, or kinesin.

78. The method of any one of claims 62-77, wherein the first, second, third, and fourth anchors are the same.

79. The method of claim 78, wherein the first, second, third, and fourth anchors comprise biotin, spectrin, a bridge protein from the ERM family, a bridge protein from the formin family, a transmembrane glycoprotein, digoxygenin, or a combination thereof.

80. The method of any one of claims 62-79, wherein the linker molecule is streptavidin, avidin, neutravidin, a biotin binding protein, an antibody directed against digoxygenin, or a combination thereof.

81. The method of any one of claims 62-80, wherein the aperture is from about 100 nanometers (nm) to about 1000 microns (μm) in diameter.

82. The method of any one of claims 62-81, wherein the substrate is a polymer resin, glass, or a semiconductor.

83. The method of any one of claims 62-82, wherein the lipid bilayer spans the aperture.

84. The method of any one of claims 62-83, wherein the polymer filaments of each of the first layer and the second layer are separated by a conduit.

85. The method of any one of claims 63-84, wherein the polymer filaments of the third layer are separated by a conduit.

86. The method of claim 84 or claim 85, wherein the conduit is from about 10⁻³ to about 10⁰ microns (μm) in diameter.

87. The method of any one of claims 62-86, wherein the lipid bilayer comprises a plurality of lipid groups comprising one or more of diphytanoyl 1,2,-diacyl-sn-glycero-3-[phosphor-L-serine] (DiPHyPC), 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE).

88. The method of any one of claims 62-87, wherein the first layer of polymer filaments, the second layer of polymer filaments, and the third layer of polymer filaments each has a density that is from about 0.01 filaments per um² to about 10,000 filaments per um².

89. The method of claim 88, wherein density of each of the first layer of polymer filaments, the second layer of polymer filaments, and the third layer of polymer filaments is about the same.

90. The method of claim 88, wherein one or more of the first layer of polymer filaments, the second layer of polymer filaments, and the third layer of polymer filaments have different densities.

91. The method of any one of claims 62-90, wherein the surface charge density of the first layer of polymer filaments, the second layer of polymer filaments, and the third layer of polymer filaments is controlled by adjusting the pH of the buffer.

92. The method of any one of claims 62-91, wherein the surface charge density of the first layer of polymer filaments, the second layer of polymer filaments, and the third layer of polymer filaments is controlled by adjusting the ionic strength of the buffer.

93. The method of any one of claims 62-92, wherein the formation success frequency is from about 70% to about 90% or more.

94. A method of forming a device comprising:
(a) providing a lipid bilayer comprising biotin, wherein the lipid bilayer is associated with a substrate, the substrate comprising an aperture and an electrode;
(b) applying avidin;
(c) providing a first layer of polymer filaments comprising biotin, thereby creating a cross-linking site between the biotin on the lipid bilayer, the avidin, and the biotin on the first layer of polymer filaments, thereby linking the lipid bilayer to the first layer of polymer filaments;
(d) applying avidin;
(e) providing a second layer of polymer filaments comprising biotin, thereby creating a cross-linking site between the biotin on the first layer of polymer filaments, the avidin, and the biotin on the second layer of polymer filaments, thereby linking the first layer of polymer filaments to the second layer of polymer filaments; and
(f) inserting a pore into the lipid bilayer, thereby forming the device.

95. The method of claim 94, further comprising applying avidin between steps (e) and (f), and
providing a third layer of polymer filaments comprising biotin, thereby creating a cross-linking site between the biotin on the second layer of polymer filaments, the avidin, and the biotin on the third layer of polymer filaments, thereby linking the second layer of polymer filaments to the third layer of polymer filaments.
